(19) United States
(12) Patent Application Publication
(10) Pub. No.: US 2012/0116095 A1
(43) Pub. Date: May 10, 2012

(54) GLYCINE TRANSPORTER INHIBITORS

(75) Inventors: Akito Yasuhara, Toshima-ku (JP); Kazunari Sakagami, Toshima-ku (JP); Hiroshi Ohta, Toshima-ku (JP); Masato Hayashi, Toshima-ku (JP); Yoshihisa Shirasaki, Toshima-ku (JP); Shuji Yamamoto, Toshima-ku (JP); Youichi Shimazaki, Toshima-ku (JP); Yuko Araki, Toshima-ku (JP); Kumi Abe, Toshima-ku (JP); Xiang-Min Sun, Toshima-ku (JP)

(73) Assignee: TAISHO PHARMACEUTICAL C0., LTD., Toshima-ku, Tokyo (JP)

(21) Appl. No.: 13/383,513

(22) PCT Filed: Jul. 15, 2010
(86) PCT No.: PCT/JP2010/062394

§ 371 Date: Jan. 11, 2012

(30) Foreign Application Priority Data
Jul. 15, 2009 (JP) 2009-166355

(51) Int. Cl. C07D 249/12; C07D 233/14

(52) U.S. Cl. 548/266.8; 548/334.1

(57) ABSTRACT

The present invention aims to provide novel compounds represented by formula [I] or pharmaceutically acceptable salts thereof:

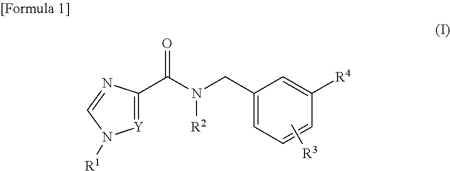

which are useful for prevention or treatment of diseases such as schizophrenia, Alzheimer's disease, cognitive impairment, dementia, anxiety disorders (e.g., generalized anxiety disorder, panic disorder, obsessive-compulsive disorder, social anxiety disorder, post-traumatic stress disorder, specific phobias, acute stress disorder), depression, drug dependence, convulsion, tremor, pain, or sleep disorders, based on their inhibitory effect against glycine uptake.

### TECHNICAL FIELD

The present invention relates to compounds having an inhibitory effect against glycine transporter.

### BACKGROUND ART

NMDA receptor, which is one of the glutamate receptors, is located on the neural cell membrane in the brain and is involved in various neurophysiological events including neuronal plasticity, cognition, attention, memory, etc. The NMDA receptor has multiple allosteric binding sites, one of which is a glycine-binding site (glycine-binding site on NMDA receptor complex). The glycine binding site on NMDA receptor complex is reported to participate in activation of NMDA receptors (NPL 1).

When an action potential reaches the presynaptic terminals of glycinergic nerves, glycine begins to be released into synaptic clefts. The released glycine binds to postsynaptic receptors, and is then removed from the synaptic clefts by its transporters. In view of this fact, glycine transporters probably regulate the amount of glycine in the extracellular fluid and thereby control functions of NMDA receptors.

Glycine transporters (GlyTs) are proteins that contribute to reuptake of extracellular glycine into cells. Until now, there have been found two subtypes, i.e., GlyT1 and GlyT2. GlyT1 is expressed primarily in the cerebral cortex, hippocampus and thalamus, etc., and is reported to be involved in diseases such as schizophrenia, Alzheimer's disease, cognitive impairment, dementia, anxiety disorders (e.g., generalized anxiety disorder, panic disorder, obsessive-compulsive disorder, social anxiety disorder, post-traumatic stress disorder, specific phobias, acute stress disorder), depression, drug dependence, convulsion, tremor, pain, and sleep disorders (NPL 2 to NPL 4).

Compounds having an inhibitory effect against GlyT1 and having a 5-membered cyclic heteroarylamide structure are reported in the patent documents listed below (PTL 1 to PTL 3). Compounds disclosed in PTL 1 to PTL 3 and those disclosed in NFL 5 and NPL 6 are characterized in that a nitrogen-containing group is attached to the nitrogen atom in the amide structure.

### CITATION LIST

### Patent Literature

PTL 1: WO2005/037216
PTL 2: WO2006/106425
PTL 3: WO2008/065500

### Non Patent Literature

NPL 1: Molecular Psychiatry (2004) 9, 984-997
NPL 2: Current Medicinal Chemistry, 2006, 13, 1017-1044
NPL 3: Neuropsychopharmacology (2005), 1-23
NPL 4: Expert Opinion on Therapeutic Patents (2004) 14 (2) 201-214
NPL 5: Bioorganic & Medicinal Chemistry Letters (2009) 19 2974-2976
NPL 6: Bioorganic & Medicinal Chemistry Letters (2010) 20 907-911

### SUMMARY OF INVENTION

### Technical Problem

The present invention aims to provide novel compounds or pharmaceutically acceptable salts thereof, which are useful for prevention or treatment of diseases such as schizophrenia, Alzheimer's disease, cognitive impairment, dementia, anxiety disorders (e.g., generalized anxiety disorder, panic disorder, obsessive-compulsive disorder, social anxiety disorder, post-traumatic stress disorder, specific phobias, acute stress disorder), depression, drug dependence, convulsion, tremor, pain, or sleep disorders, based on their inhibitory effect against glycine uptake.

### Solution to Problem

As a result of extensive and intensive studies on structurally novel compounds with an inhibitory effect against GlyT1, the inventors of the present invention have found that compounds represented by the following formula are excellent GlyT1 inhibitors. This finding led to the completion of the present invention.

The present invention will be described in more detail below. Embodiments of the present invention (hereinafter each referred to as “the compound of the present invention”) are as shown below.

(1) A compound represented by formula [I] or a pharmaceutically acceptable salt thereof:

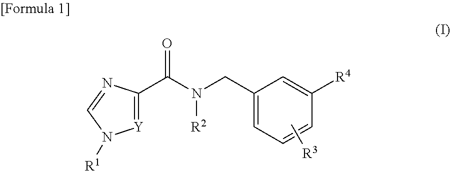

wherein

R¹ represents a hydrogen atom or a C₁₋₆ alkyl group,

R² represents a C₁₋₆ alkyl group, a C₂₋₆ alkenyl group, a C₃₋₆ cycloalkyl group, a C₁₋₆ haloalkyl group, a C₁₋₆ hydroxyalkyl group, or a C₁₋₆ alkoxy-C₁₋₆ alkyl group,

R³ represents a hydrogen atom, a C₁₋₆ alkyl group, a C₁₋₆ alkoxy group, a C₁₋₆ haloalkyl group, a C₁₋₆ haloalkoxy group, or a halogen atom,

R⁴ represents a phenyl group, which may be substituted with 1 to 3 substituents selected from the group consisting of a C₁₋₆ alkyl group, a C₁₋₆ alkoxy group, a C₁₋₆ haloalkyl group, a C₁₋₆ haloalkoxy group, a C₁₋₆ hydroxyalkyl group, a C₁₋₆ alkoxy-C₁₋₆ alkyl group, a C₂₋₇ alkoxycarbonyl group, a cyano group, and a halogen atom, and

Y represents the formula CH or a nitrogen atom.

(2) The compound or pharmaceutically acceptable salt thereof according to (1) above, wherein R² is a branched C₃₋₆ alkyl group or a C₃₋₆ cycloalkyl group.
(3) The compound or pharmaceutically acceptable salt thereof according to (1) or (2) above, wherein Y is the formula CH.
(4) The compound or pharmaceutically acceptable salt thereof according to any one of (1) to (3) above, wherein R³ is a halogen atom.
(5) The compound or pharmaceutically acceptable salt thereof according to any one of (1) to (3) above, wherein R³ is a fluorine atom.
(6) The compound or pharmaceutically acceptable salt thereof according to any one of (1) to (5) above, wherein R⁴ is a phenyl group, or a phenyl group substituted with 1 to 3 substituents selected from the group consisting of a C₁₋₆ alkoxy group, a C₁₋₆ haloalkoxy group, a C₁₋₆ hydroxyalkyl group, and a halogen atom.
(7) The compound or pharmaceutically acceptable salt thereof according to (1) above,
wherein the compound of formula [I] is represented by the following formula:

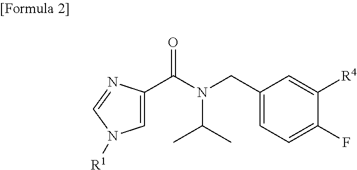

wherein R¹ represents a methyl group or an ethyl group, and R⁴ is a phenyl group, or a phenyl group substituted with 1 to 3 substituents selected from the group consisting of a C₁₋₆ alkoxy group, a C₁₋₆ haloalkoxy group, a C₁₋₆ hydroxyalkyl group, and a halogen atom.
(8) The compound or pharmaceutically acceptable salt thereof according to (1) above,

wherein the compound is
N-{[6-fluoro-4′-(trifluoromethoxy)biphenyl-3-yl]methyl}-1-methyl-N-(propan-2-yl)-1H-imidazole-4-carboxamide,
N-{[6-fluoro-3′-(trifluoromethoxy)biphenyl-3-yl]methyl}-1-methyl-N-(propan-2-yl)-1H-imidazole-4-carboxamide,
1-methyl-N-(propan-2-yl)-N-[(3′,4′,6-trifluorobiphenyl-3-yl)methyl]-1H-imidazole-4-carboxamide,
N-[(4′,6-difluorobiphenyl-3-yOmethyl]-1-methyl-N-(propan-2-yl)-1H-imidazole-4-carboxamide,
N-[(3′,6-difluoro-4′-methoxybiphenyl-3-yl)methyl]-1-methyl-N-(propan-2-yl)-1H-imidazole-4-carboxamide,
N-[(3′,6-difluorobiphenyl-3-yl)methyl]-1-methyl-N-(propan-2-yl)-1H-imidazole-4-carboxamide,
N-[(6-fluorobiphenyl-3-yl)methyl]-1-methyl-N-(propan-2-yl)-1H-imidazole-4-carboxamide,
N-[(6-fluoro-3′-methoxybiphenyl-3-yl)methyl]-1-methyl-N-(propan-2-yl)-1H-imidazole-4-carboxamide,
N-[(6-fluoro-4′-methoxybiphenyl-3-yl)methyl]-1-methyl-N-(propan-2-yl)-1H-imidazole-4-carboxamide,
N-[(4′-chloro-6-fluorobiphenyl-3-yl)methyl]-1-methyl-N-(propan-2-yl)-1H-imidazole-4-carboxamide,
1-methyl-N-(propan-2-yl)-N-[(3′,5′,6-trifluorobiphenyl-3-yl)methyl]-1H-imidazole-4-carboxamide,
N-{[3′-(difluoromethoxy)-6-fluorobiphenyl-3-yl]methyl}-1-methyl-N-(propan-2-yl)-1H-imidazole-4-carboxamide,
N-{[4′-(difluoromethoxy)-6-fluorobiphenyl-3-yl]methyl}-1-methyl-N-(propan-2-yl)-1H-imidazole-4-carboxamide,
1-methyl-N-(propan-2-yl)-N-[(3′,4′,5′,6-tetrafluorobiphenyl-3-yl)methyl]-1H-imidazole-4-carboxamide, or
1-ethyl-N-{[6-fluoro-4′-(trifluoromethoxy)biphenyl-3-yl]methyl}-N-(propan-2-yl)-1H-imidazole-4-carboxamide.
   (9) The compound or pharmaceutically acceptable salt thereof according to (1) above, wherein the compound is
N-{[6-fluoro-4′-(trifluoromethoxy)biphenyl-3-yl]methyl}-1-methyl-N-(propan-2-yl)-1H-imidazole-4-carboxamide,
N-{[6-fluoro-3′-(trifluoromethoxy)biphenyl-3-yl]methyl}-1-methyl-N-(propan-2-yl)-1H-imidazole-4-carboxamide,
1-methyl-N-(propan-2-yl)-N-[(3′,4′,6-trifluorobiphenyl-3-yl)methyl]-1H-imidazole-4-carboxamide,
N-[(4′,6-difluorobiphenyl-3-yl)methyl]-1-methyl-N-(propan-2-yl)-1H-imidazole-4-carboxamide,
N-{[3′-(difluoromethoxy)-6-fluorobiphenyl-3-yl]methyl}-1-methyl-N-(propan-2-yl)-1H-imidazole-4-carboxamide,
N-{[4′-(difluoromethoxy)-6-fluorobiphenyl-3-yl]methyl}-1-methyl-N-(propan-2-yl)-1H-imidazole-4-carboxamide,
1-methyl-N-(propan-2-yl)-N-[(3′,4′,5′,6-tetrafluorobiphenyl-3-yl)methyl]-1H-imidazole-4-carboxamide, or
1-ethyl-N-{[6-fluoro-4′-(trifluoromethoxy)biphenyl-3-yl]methyl}-N-(propan-2-yl)-1H-imidazole-4-carboxamide.
   (10) A pharmaceutical preparation, which comprises the compound or pharmaceutically acceptable salt thereof according to any one of (1) to (9) above as an active ingredient.
   (11) A prophylactic or therapeutic agent for diseases of schizophrenia, Alzheimer's disease, cognitive impairment, dementia, anxiety disorders, depression, drug dependence, convulsion, tremor, or sleep disorders, which comprises the compound or pharmaceutically acceptable salt thereof according to any one of (1) to (9) above as an active ingredient.

### ADVANTAGEOUS EFFECTS OF INVENTION

The compounds of the present invention have inhibitory activity against glycine transporter (GlyT1). Moreover, as shown in the test examples described later, the compounds of the present invention also have high membrane permeability and hence are expected to have superior intestinal absorption, which is an important property for orally administered drugs. Furthermore, as shown in the test examples described later, the compounds of the present invention are not recognized as substrates for P-glycoprotein, which is an efflux transporter that controls brain penetration of drugs, and hence are expected to have good brain penetration.

### DESCRIPTION OF EMBODIMENTS

As used herein, the term “C₁₋₆ alkyl group” is intended to mean a linear or branched alkyl group containing 1 to 6 carbon atoms. Examples include methyl, ethyl, propyl, isopropyl, butyl, isobutyl, tert-butyl, pentyl, isopentyl, and hexyl.

As used herein, the term “C₂₋₆ alkenyl group” is intended to mean a linear or branched alkenyl group containing 2 to 6 carbon atoms. Examples include vinyl, allyl, but-2-enyl, and prop-1-en-2-yl.

As used herein, the term “C₃₋₆ cycloalkyl group” is intended to mean a cycloalkyl group containing 3 to 6 carbon atoms, i.e., cyclopropyl, cyclobutyl, cyclopentyl, and cyclohexyl.

As used herein, the term “C₁₋₆ alkoxy group” is intended to mean a linear or branched alkoxy group containing 1 to 6 carbon atoms. Examples include methoxy, ethoxy, propoxy, isopropoxy, butoxy, isobutoxy, pentyloxy, isopentyloxy, and hexyloxy.

As used herein, the term “C₁₋₆ alkoxy-C₁₋₆ alkyl group” is intended to mean a C₁₋₆ alkyl group substituted with a C₁₋₆ alkoxy group(s). Examples include methoxymethyl, methoxyethyl, methoxypropyl, methoxybutyl, methoxypentyl, methoxyhexyl, ethoxymethyl, ethoxyethyl, ethoxypropyl, isopropoxymethyl, isopropoxyethyl, isopropoxypropyl, 1-methoxyethyl, 1-ethoxyethyl, 2-methoxypropyl, and 2-ethoxypropyl.

As used herein, the term “halogen (halo)” refers to a fluorine atom, a chlorine atom, a bromine atom, or an iodine atom.

As used herein, the term “C₁₋₆ haloalkyl group” is intended to mean a linear or branched C₁₋₆ alkyl group substituted with a halogen atom(s), preferably with 1 to 3 halogen atoms. Examples include fluoromethyl, difluoromethyl, trifluoromethyl, and trichloromethyl.

As used herein, the term “C₁₋₆ haloalkoxy group” is intended to mean a linear or branched C₁₋₆ alkoxy group substituted with a halogen atom(s), preferably with 1 to 3 halogen atoms. Examples include fluoromethoxy, difluoromethoxy, and trifluoromethoxy.

As used herein, the term “C₁₋₆ hydroxyalkyl group” is intended to mean a linear or branched C₁₋₆ alkyl group substituted with a hydroxy group(s). Examples include hydroxymethyl, 2-hydroxyethyl, 1-hydroxyethyl, 3-hydroxypropyl, 2-hydroxypropyl, and 1-hydroxypropyl.

As used herein, the term “C₂₋₇ alkoxycarbonyl group” refers to a linear or branched alkoxycarbonyl group containing 2 to 7 carbon atoms. Examples include methoxycarbonyl, ethoxycarbonyl, propoxycarbonyl, isopropoxycarbonyl, butoxycarbonyl, and isobutoxycarbonyl.

As used herein, the term “pharmaceutically acceptable salt” is intended to mean a pharmaceutically acceptable acid addition salt. Examples of an acid to be used for this purpose include inorganic acids such as sulfuric acid, hydrochloric acid, hydrobromic acid, nitric acid and phosphoric acid, as well as organic acids such as acetic acid, oxalic acid, lactic acid, citric acid, malic acid, gluconic acid, tartaric acid, fumaric acid, maleic acid, methanesulfonic acid, ethanesulfonic acid, benzenesulfonic acid and p-toluenesulfonic acid. The compounds in free form can be converted into these salts in a conventional manner.

Preferred embodiments will be given below for the compounds of the present invention.

R² is preferably a branched C₃₋₆ alkyl group or a C₃₋₆ cycloalkyl group, more preferably a branched C₃₋₆ alkyl group.

R³ is preferably a halogen atom, more preferably a fluorine atom. In the case of a fluorine atom as a more preferred example of R³, the structure of formula (I) is more preferably represented by the following formulae:

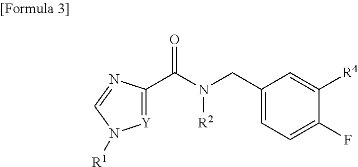

R¹ is preferably a C₁₋₆ alkyl group, more preferably a methyl group or an ethyl group, even more preferably a methyl group.

R⁴ is preferably a phenyl group, or a phenyl group substituted with 1 to 3 substituents selected from the group consisting of a C₁₋₆ alkoxy group, a C₁₋₆ haloalkoxy group, a C₁₋₆ hydroxyalkyl group, and a halogen atom.

The compounds of the present invention may contain multiple asymmetric centers. Thus, the above compounds can also exist not only in optically active form but also as racemates. Further, there may be a plurality of diastereomers. All of these forms fall within the scope of the present invention. Individual isomers can be obtained in a known manner, for example, by using optically active starting materials or intermediates, by using optically selective reaction or diastereoselective reaction in the preparation of intermediates or final products, or by using chromatographic separation in the preparation of intermediates or final products. Further, if the compounds of the present invention form hydrates or solvates, these hydrates or solvates also fall within the scope of the present invention. Likewise, pharmaceutically acceptable salts of hydrates or solvates of the compounds of the present invention also fall within the scope of the present invention.

Other embodiments of the present invention are as shown below.

(A) A compound represented by formula [I] or a pharmaceutically acceptable salt thereof:

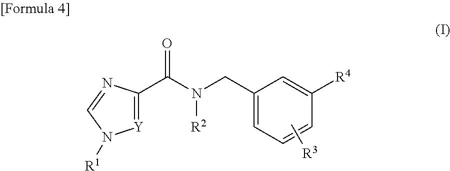

wherein

R¹ represents a hydrogen atom or a C₁₋₆ alkyl group,

R² represents a C₁₋₆ alkyl group, a C₃₋₆ cycloalkyl group, a C₁₋₆ haloalkyl group, or a C₁₋₆ alkoxy-C₁₋₆ alkyl group,

R³ is a hydrogen atom or a halogen atom, and

R⁴ represents a phenyl group, which may be substituted with 1 to 3 substituents selected from a C₁₋₆ alkyl group, a C₁₋₆ alkoxy group, a C₁₋₆ haloalkyl group, a C₁₋₆ haloalkoxy group, a C₁₋₆ hydroxyalkyl group, a C₁₋₆ alkoxy-C₁₋₆ alkyl group, a C₂₋₆ alkoxycarbonyl group, a cyano group, and a halogen atom.

Y represents a nitrogen atom or the formula CH.

(B) The compound or pharmaceutically acceptable salt thereof according to (A) above, wherein R¹ is a C₁₋₆ alkyl group, and Y is the formula CH.
(C) The compound or pharmaceutically acceptable salt thereof according to (A) above, wherein R¹ is a C₁₋₆ alkyl group, and Y is a nitrogen atom.
(D) The compound or pharmaceutically acceptable salt thereof according to any one of (A) to (C) above, wherein R³ is a halogen atom.
(E) The compound or pharmaceutically acceptable salt thereof according to any one of (A) to (C) above, wherein R³ is a fluorine atom.
(F) The compound or pharmaceutically acceptable salt thereof according to any one of (A) to (E) above, wherein R² is a C₁₋₆ alkyl group or a C₃₋₆ cycloalkyl group.
(G) The compound or pharmaceutically acceptable salt thereof according to any one of (A) to (E) above, wherein R² is a C₁₋₆ alkoxy-C₁₋₆ alkyl group.

The compounds of the present invention can be administered orally or parenterally in dosage forms such as tablets, capsules, granules, powders, troches, ointments, creams, emulsions, suspensions, suppositories, injections or the like, all of which may be prepared according to conventional formulation techniques (e.g., the procedures defined in the 15th revised Japanese Pharmacopoeia). These dosage forms may be selected as appropriate depending on the symptom and age of a patient as well as the aim of therapy.

To prepare these preparations, a composition containing the compound of the present invention may be blended with pharmacologically acceptable carriers, i.e., excipients (e.g., crystalline cellulose, starch, lactose, mannitol), binders (e.g., hydroxypropylcellulose, polyvinylpyrrolidone), lubricants (e.g., magnesium stearate, talc), disintegrants (e.g., carboxymethylcellulose calcium) and/or various other pharmacologically acceptable additives.

Moreover, the compounds of the present invention may be used in combination with one or more other therapeutic agents, various antipsychotics, antidepressants, for example, 5HT3 antagonists, 5HT2 antagonists, serotonin agonists, NK-1 antagonists, selective serotonin reuptake inhibitors (SSRI), serotonin-noradrenaline reuptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists, anticonvulsants, cognitive function enhancers, and other psychoactive drugs.

Examples of other therapeutic agents that may be used in combination with the compounds of the present invention include ondansetron, granisetron, metoclopramide, sumatriptan, rauwolscine, yohimbine, metoclopramide, fluoxetine, citalopram, escitalopram, femoxetine, fluvoxamine, paroxetine, indalpine, sertraline, zimeldine, venlafaxine, reboxetine, Milnacipran, duloxetine, imipramine, amitriptiline, chlomipramine, nortriptiline, bupropion, amineptine, divalproex, carbamazepine, diazepam, risperidone, olanzapine, ziprasidone, aripiprazole, quetiapine, perospirone, clozapine, haloperidol, pimozide, droperidol, chlorpromazine, thioridazine, mesoridazine, trifluoperazine, perphenazine, fluphenazine, thiflupromazine, prochlorperazine, acetophenazine, thiothixene, chlorprothixene, lamotrigine, loxapine, molindone, and so on. These combinations may be administered simultaneously (in a single dosage form or in separate dosage forms), separately, or successively.

Use and therapy in combination with the compounds of the present invention are particularly advantageous in that equal or improved efficacy can be achieved by using the respective ingredients at lower doses than their usual doses, and are also expected to further enhance the therapeutic effects on positive and/or negative symptoms of mental disorders and/or cognitive impairment. Use and therapy in combination with the compounds of the present invention may also provide benefits in treating patients who are not sufficiently responsive to treatment with some type of neuroleptic or who are resistant to such treatment.

For use in adults, the daily dosage of the compound of the present invention is 1 to 2000 mg, given as a single dose or in divided doses per day. This dosage may be increased or decreased as appropriate for the age, body weight and symptom of a patient.

The compounds of formula [I] can be prepared by various synthesis procedures. The procedures shown below are given as examples of how to prepare the compounds of the present invention, and the present invention is not limited thereto.

In the general preparation procedures shown below, the term “inert solvent” refers to, for example, an alcohol (e.g., methanol, ethanol, isopropanol, n-butanol, ethylene glycol), an ether (e.g., diethyl ether, t-butyl methyl ether, diisopropyl ether, tetrahydrofuran, 1,4-dioxane, 1,2-dimethoxyethane), a hydrocarbon (e.g., pentane, hexane, heptane, toluene, benzene, xylene), an ester (e.g., ethyl acetate, ethyl formate), a ketone (e.g., acetone, methyl ethyl ketone), a halogenated carbon solvent (e.g., chloroform, dichloromethane), an amide (e.g., dimethylformamide, N-methylpyrrolidone), acetonitrile, dimethyl sulfoxide, water, or any mixed solvent thereof, etc.

The term “base” refers to, for example, a hydride of an alkali or alkaline earth metal (e.g., lithium hydride, sodium hydride, potassium hydride, calcium hydride); an amide of an alkali or alkaline earth metal (e.g., lithium amide, sodium amide, lithium diisopropylamide, lithium dicyclohexylamide, lithium hexamethyldisilazide, sodium hexamethyldisilazide, potassium hexamethyldisilazide); a lower alkoxide of an alkali or alkaline earth metal (e.g., sodium methoxide, sodium ethoxide, potassium tert-butoxide); an alkyllithium (e.g., butyllithium, sec-butyllithium, tert-butyllithium, methyllithium); a hydroxide of an alkali or alkaline earth metal (e.g., sodium hydroxide, potassium hydroxide, lithium hydroxide, barium hydroxide); a carbonate salt of an alkali or alkaline earth metal (e.g., sodium carbonate, potassium carbonate, cesium carbonate); a bicarbonate salt of an alkali or alkaline earth metal (e.g., sodium bicarbonate, potassium bicarbonate); an amine (e.g., triethylamine, N-methylmorpholine, N,N-diisopropylethylamine, 1,8-diazabicyclo[5.4.0]undec-7-ene (DBU), 1,5-diazabicyclo[4.3.0]non-5-ene (DBN), N,N-dimethylaniline); a basic heterocyclic compound (e.g., pyridine, imidazole, 2,6-lutidine), etc. These bases are selected as appropriate for various reaction conditions known to those skilled in the art.

The term “acid” refers to, for example, an inorganic acid (e.g., hydrochloric acid, hydrobromic acid, sulfuric acid, nitric acid, phosphoric acid) or an organic acid (e.g., p-toluenesulfonic acid, methanesulfonic acid, trifluoroacetic acid, formic acid, acetic acid, citric acid, oxalic acid). These acids are selected as appropriate for various reaction conditions known to those skilled in the art.

The term “Lewis acid” is intended to include, for example, boron trifluoride, aluminum trichloride, titanium tetrachloride, iron trichloride, zinc chloride, tin tetrachloride and so on.

General Preparation Procedure 1

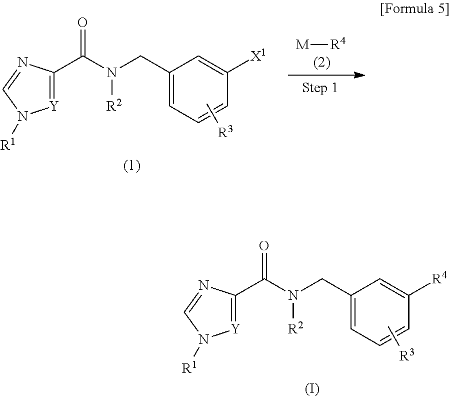

wherein X¹ represents a bromine atom, an iodine atom, or a trifluoromethanesulfonyloxy group, and the other symbols are as defined above.

Step 1: In an inert solvent and in the presence or absence of a base, compound (1) may be reacted with compound (2) by using a palladium catalyst and, if necessary, a ligand of the palladium catalyst to obtain the compound (1) of the present invention.

In this step, examples of the palladium catalyst include palladium acetate, tris(dibenzylideneacetone)dipalladium, tetrakis(triphenylphosphine) palladium, dichlorobis(triphenylphosphine)palladium, (1,3-diisopropylimidazol-2-ylidene)(3-chloropyridyl)palladium(II) dichloride, [1,3-bis(2,6-diisopropylphenyl)imidazol-2-ylidene](3-chloropyridyl)palladium(II) dichloride, [1,1′-bis(diphenylphosphino)ferrocene]palladium chloride and so on. Examples of the ligand include triphenylphosphine, 2,2-bis(diphenylphosphino)-1,1-binaphthyl (BINAP), 2-(di-tert-butylphosphino)biphenyl, 9,9-dimethyl-4,5-bis(diphenylphosphino)xanthene (Xantphos) and so on. Examples of compound (2) include Grignard reagents (e.g., R⁴MgCl), zinc reagents (e.g., R⁴ZnCl), boron reagents (e.g., those in which R⁴ is attached to boric acid or a boric acid ester), tin reagents (e.g., R⁴SnBu₃) and so on.

General Preparation Procedure 2

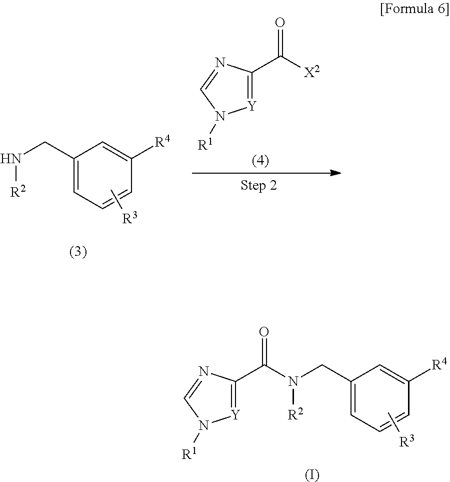

wherein X² represents a halogen atom or a hydroxy group, and the other symbols are as defined above.

Step 2: In an inert solvent and in the presence or absence of a base, compound (3) may be reacted with compound (4) in which X² is a halogen atom to obtain the compound (1) of the present invention. Alternatively, compound (3) and compound (4) in which X² is a hydroxyl group may be subjected to various types of amidation reactions known to those skilled in the art to obtain the compound (1) of the present invention. In this step, such amidation reactions include an amidation reaction in an inert solvent and in the presence or absence of a base using a condensing agent such as O-(7-azabenzotriazol-1-yl)-N,N,N′,N′-tetramethyluronium hexafluorophosphate (HATU), O-(benzotriazol-1-yl)-N,N,N′,N′-tetramethyluronium hexafluorophosphate (HBTU), N,N′-dicyclohexylcarbodiimide (DCC), 1-ethyl-3-(3-dimethylaminopropyl)carbodiimide hydrochloride (EDC.HCl), diphenylphosphorylazide (DPPA) or carbonyldiimidazole (CDI), as well as an amidation reaction via a mixed acid anhydride using ethyl chlorocarbonate, isobutyl chlorocarbonate, trimethylacetyl chloride or the like. In the case of the amidation reaction using a condensing agent, it is possible to use an additive such as 1-hydroxybenzotriazole (HOBt) or hydroxysuccinimide (HOSu), if necessary.

General Preparation Procedure 3

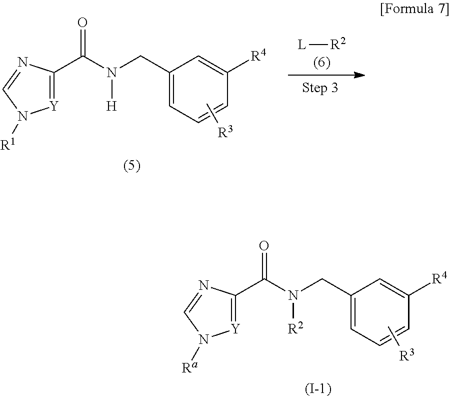

wherein L represents a leaving group such as a halogen atom, a methanesulfonyloxy group, a trifluoromethanesulfonyloxy group or a p-toluenesulfonyloxy group, R^{a} represents a C₁₋₆ alkyl group, and the other symbols are as defined above.

Step 3: In an inert solvent and in the presence or absence of a base, compound (5) may be reacted with compound (6) to obtain the compound (1-1) of the present invention.

General Preparation Procedure 4

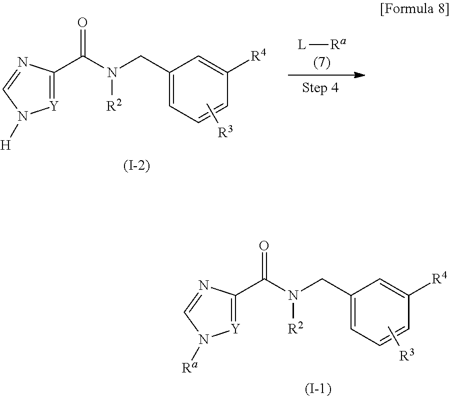

wherein the symbols are as defined above.

Step 4: In an inert solvent and in the presence or absence of a base, the compound (1-2) of the present invention may be reacted with compound (7) to obtain the compound (1-1) of the present invention.

General Preparation Procedure 5

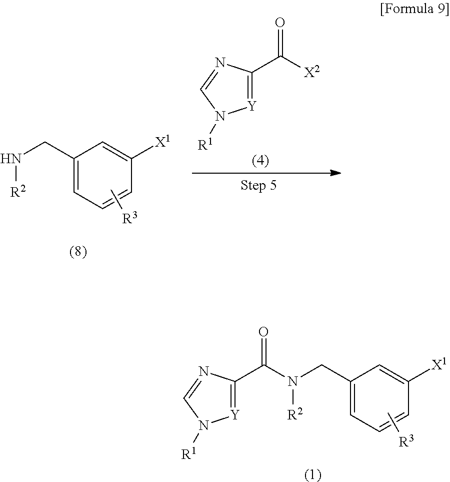

Step 5: Starting from compound (8) and compound (4), compound (1) can be synthesized in the same manner as shown in Step 2 of General Preparation Procedure 2.

General Preparation Procedure 6

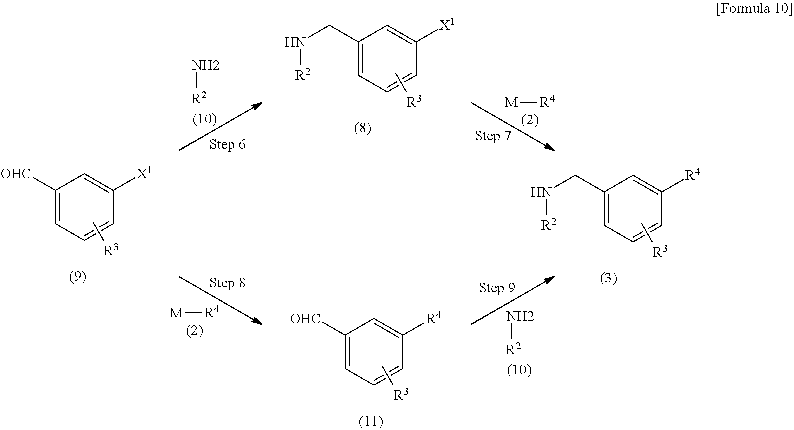

Step 6: In an inert solvent and in the presence or absence of an acid, compound (9) and compound (10) may be subjected to reductive amination reaction using a reducing agent to obtain compound (8). In this step, examples of the reducing agent include sodium triacetoxyborohydride, sodium cyanoborohydride, sodium borohydride and so on.

Step 7: Starting from compound (8) and compound (2), compound (3) can be synthesized in the same manner as shown in Step 1 of General Preparation Procedure 1.

Step 8: Starting from compound (9) and compound (2), compound (11) can be synthesized in the same manner as shown in Step 1 of General Preparation Procedure 1.

Step 9: Starting from compound (11) and compound (10), compound (3) can be synthesized in the same manner as shown in Step 6 of General Preparation Procedure 6.

General Preparation Procedure 7

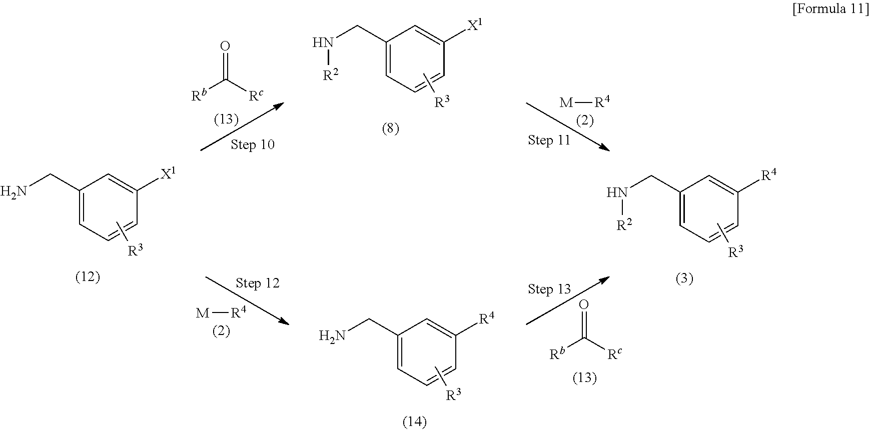

wherein R^{b} and R^{c} each independently represent a hydrogen atom, a C₁₋₆ alkyl group, a C₁₋₆ haloalkyl group, or a C₁₋₆ alkoxy-C₁₋₆ alkyl group, or alternatively, R^{b} and R^{c} may form a C₃₋₆ cycloalkyl ring together with the carbon atom to which they are attached.

Step 10: Starting from compound (12) and compound (13), compound (8) can be synthesized in the same manner as shown in Step 6 of General Preparation Procedure 6.

Step 11: Starting from compound (8) and compound (2), compound (3) can be synthesized in the same manner as shown in Step 1 of General Preparation Procedure 1.

Step 12: Starting from compound (12) and compound (2), compound (14) can be synthesized in the same manner as shown in Step 1 of General Preparation Procedure 1.

Step 13: Starting from compound (14) and compound (13), compound (3) can be synthesized in the same manner as shown in Step 6 of General Preparation Procedure 6.

General Preparation Procedure 8

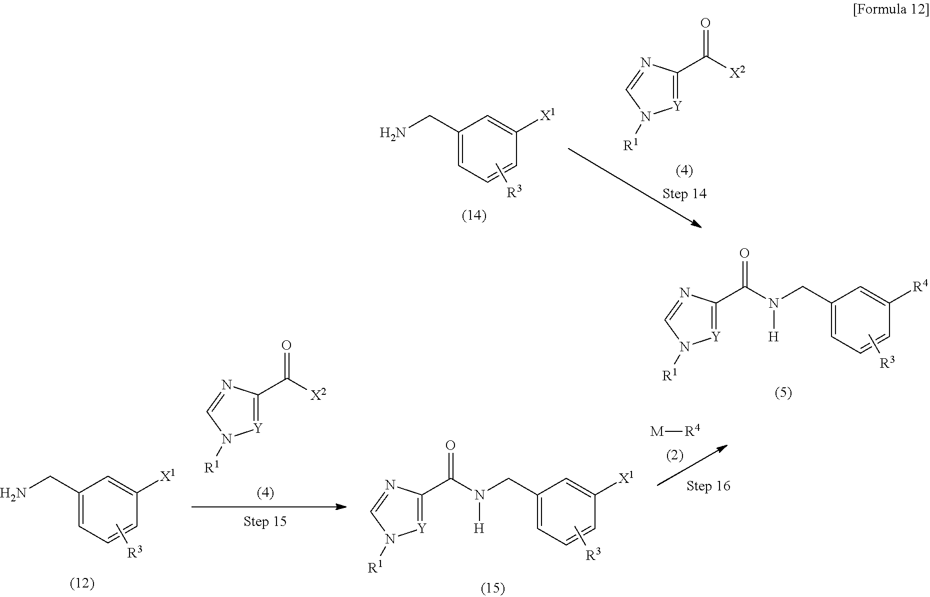

Step 14: Starting from compound (14) and compound (4), compound (5) can be synthesized in the same manner as shown in Step 2 of General Preparation Procedure 2.

Step 15: Starting from compound (12) and compound (4), compound (15) can be synthesized in the same manner as shown in Step 2 of General Preparation Procedure 2.

Step 16: Starting from compound (15) and compound (2), compound (5) can be synthesized in the same manner as shown in Step 1 of General Preparation Procedure 1.

### EXAMPLES

Next, the present invention will be further described in more detail below by way of preparation examples, examples and test examples, which are not intended to limit the scope of the present invention.

The “NH silica gel cartridge” and “silica gel cartridge” used for purification by column chromatography were a Biotage® SNAPCartridge KP-NH and a Biotage® SNAPCartridge KP-Sil, respectively.

The analytical data in the preparation examples and examples were measured by the analytical instruments listed below.

MS spectrum: SHIMADZU LCMS-2010EV or micromass Platform LC NMR spectrum: [¹H-NMR] 600 MHz: JNM-ECA600 (JEOL Ltd., Japan), 500 MHz: JNM-ECA500 (JEOL Ltd., Japan), 300 MHz: UNITYNOVA300 (Varian Inc.), 200 MHz: GEMINI2000/200 (Varian Inc.)

The microwave reaction apparatus used in the examples was an Initiator (Biotage AB).

The compound names in the examples were designated by the use of ACD/Name (ACD/Labs 10.01, Advanced Chemistry Development Inc.).

### Preparation Example 1

### 6-Fluoro-3′-(hydroxymethyl)biphenyl-3-carbaldehyde

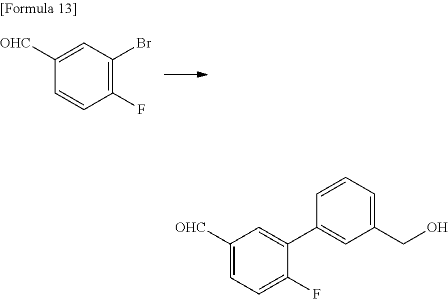

A mixture of 3-bromo-4-fluoro-benzaldehyde (1.00 g), 3-(hydroxymethyl)phenylboronic acid (787 mg), tetrakis(triphenylphosphine) palladium (569 mg), potassium carbonate (1.36 g), dimethylformamide (6 mL) and ethanol (3 mL) was reacted in a microwave reaction apparatus (150° C., 20 minutes). After addition of ethyl acetate and filtration through Celite® pad, the ethyl acetate solution was washed with water. The ethyl acetate layer was dried over with anhydrus sodium sulfate. After filtering off the desiccant, the ethyl acetate layer was concentrated under reduced pressure. The resulting residue was purified by column chromatography (silica gel cartridge, hexane:ethyl acetate=80:20 to 65:35) to give the titled compound (1.10 g).

1H NMR (600 MHz, CHLOROFORM-d) d ppm 1.74-1.81 (m, 1H) 4.74-4.82 (m, 2H) 7.28-7.62 (m, 5H) 7.85-7.91 (m, 1H) 7.97-8.04 (m, 1H) 10.0 (s, 1H)

### Preparation Example 2

### {5′-[(Cyclobutylamino)methyl]-2′-fluorobiphenyl-3-yl}methanol

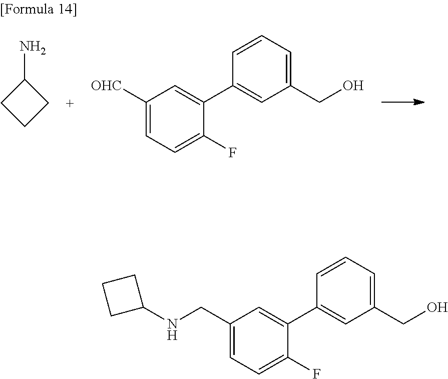

A mixture of cyclobutylamine (370 mg), 6-fluoro-3′-(hydroxymethyl)biphenyl-3-carbaldehyde (1.00 g) and chloroform (10 mL) was stirred at room temperature for 30 minutes. Sodium triacetoxyborohydride (1.29 g) was added to the mixture and stirred for 1.5 days. The reaction mixture was washed with 1 M aqueous sodium hydroxide and then dried over anhydrous sodium sulfate. After filtering off the desiccant, the solvent was distilled off under reduced pressure. The resulting residue was purified by column chromatography (silica gel cartridge, hexane:ethyl acetate=75:25 to 15:85) to give the titled compound (1.16 g).

1H NMR (600 MHz, CHLOROFORM-d) d ppm 1.59-1.79 (m, 4 μl) 2.18-2.27 (m, 2H) 3.31 (m, 1H) 3.72 (s, 2H) 4.76 (s, 2H) 7.03-7.14 (m, 1H) 7.23-7.30 (m, 1H) 7.33-7.58 (m, 5H)

(ESI pos.) m/z: 286 ([M+H]⁺)

The following compounds were synthesized by the same procedure.

### {5′-[(Cyclopentylamino)methyl]-2′-fluorobiphenyl-3-yl}methanol

1H NMR (600 MHz, CHLOROFORM-d) d ppm 1.31-1.43 (m, 2H) 1.50-1.59 (m, 2H) 1.65-1.74 (m, 2H) 1.82-1.91 (m, 2H) 3.09-3.17 (m, 1H) 3.77 (s, 2H) 4.75 (s, 2H) 7.05-7.13 (m, 1H) 7.22-7.29 (m, 1H) 7.33-7.58 (m, 5H)

(ESI pos.) m/z: 300 ([M+H]⁺)

{2′-Fluoro-5′-[(pentan-3-ylamino)methyl]biphenyl-3-yl}methanol

1H NMR (600 MHz, CHLOROFORM-d) d ppm 0.83-0.93 (m, 6 μl) 1.40-1.53 (m, 4H) 2.41-2.47 (m, 1H) 3.77 (s, 2H) 4.75 (s, 2H) 7.04-7.57 (m, 7H)

(ESI pos.) m/z: 302 ([M+H]⁺)

### Preparation Example 3

### N-(3-Bromo-4-fluorobenzyl)-2-methoxyethanamine

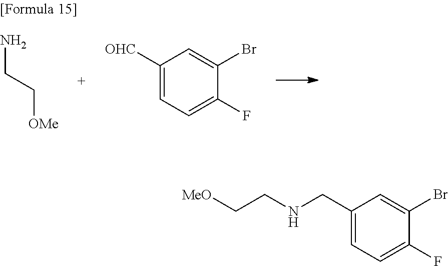

A mixture of 2-methoxyethylamine (337 mg), 3-bromo-4-fluoro-benzaldehyde (1.00 g) and chloroform (10 mL) was stirred at room temperature for 30 minutes. Sodium triacetoxyborohydride (1.33 g) was added to the mixture and reacted for 12 hours. The reaction mixture was washed with 1 M aqueous sodium hydroxide and then dried over anhydrous sodium sulfate. After filtering off the desiccant, the filtrate was evaporated under reduced pressure. The resulting residue was purified by column chromatography (silica gel cartridge, chloroform:methanol=100:0 to 97:3) to give the titled compound (796 mg).

1H NMR (600 MHz, CHLOROFORM-d) d ppm 2.75-2.80 (m, 2H) 3.36 (s, 3H) 3.48-3.53 (m, 2H) 3.76 (s, 2 μl) 7.03-7.08 (m, 1H) 7.21-7.25 (m, 1H) 7.53-7.56 (m, 1H)

(ESI pos.) m/z: 262, 264 ([M+H]⁺)

The following compounds were synthesized by the same procedure

### N-(3-Bromo-4-fluorobenzyl)cyclopentanamine

1H NMR (600 MHz, CHLOROFORM-d) d ppm 1.31-1.39 (m, 2H) 1.51-1.59 (m, 2H) 1.65-1.75 (m, 2H) 1.81-1.89 (m, 2H) 3.05-3.13 (m, 1H) 3.72 (s, 2H) 7.02-7.08 (m, 1H) 7.20-7.25 (m, 1H) 7.51-7.55 (m, 1H)

(ESI pos.) m/z: 272, 274 ([M+H]⁺)

### N-(3-Bromo-4-fluorobenzyl)-2-methylpropan-1-amine

1H NMR (600 MHz, CHLOROFORM-d) d ppm 0.92 (d, J=6.88 Hz, 6H) 1.70-1.80 (m, 1H) 2.41 (d, J=6.88 Hz, 2H) 3.73 (s, 2H) 7.06 (t, J=8.48 Hz, 1H) 7.21-7.25 (m, 1H) 7.52-7.56 (m, 1H)

(ESI pos.) m/z: 260, 262 ([M+H]⁺)

### N-(3-Bromo-4-fluorobenzyl)propan-2-amine

1H NMR (600 MHz, CHLOROFORM-d) d ppm 1.02-1.11 (m, 6H) 2.77-2.90 (m, 1H) 3.73 (s, 2H) 7.00-7.08 (m, 1H) 7.20-7.25 (m, 1H) 7.47-7.55 (m, 1H)

(ESI pos.) m/z: 246, 248 ([M+H]⁺)

### N-(3-Bromo-4-fluorobenzyl)cyclohexanamine

1H NMR (600 MHz, CHLOROFORM-d) d ppm 1.01-1.32 (m, 5 μl) 1.57-1.92 (m, 5H) 2.36-2.48 (m, 1H) 3.53 (s, 2H) 6.98-7.03 (m, 1H) 7.17-7.21 (m, 1H) 7.47 (dd, J=6.65, 2.06 Hz, 1H)

(ESI pos.) m/z: 286, 288 ([M+H]⁺)

### 2-[(3-Bromo-4-fluorobenzyl)amino]propan-1-ol

1H NMR (600 MHz, CHLOROFORM-d) d ppm 1.12 (d, J=6.42 Hz, 3H) 2.85-2.93 (m, 1H) 3.33-3.41 (m, 1H) 3.61-3.94 (m, 4H) 7.06-7.11 (m, 1H) 7.27-7.29 (m, 1H) 7.55-7.58 (m, 1H)

(ESI pos.) m/z: 262, 264 ([M+H]⁺)

### Preparation Example 4

### N-(3-Bromo-4-fluorobenzyl)-N-cyclopentyl-1-methyl-1H-imidazole-4-carboxamide

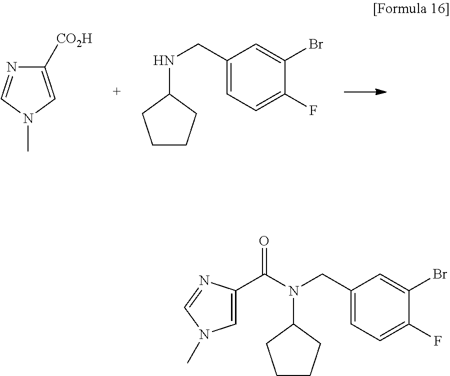

A mixture of N-(3-bromo-4-fluorobenzyl)cyclopentanamine (1.50 g), 1-methyl-1H-imidazole-4-carboxylic acid (695 mg), 2-(1H-7-azabenzotriazol-1-yl)-1,1,3,3-tetramethyl uronium hexafluorophosphate methanaminium (HATU) (2.72 g), diisopropylethylamine (2.55 mL) and acetonitrile (23 mL) was stirred for 4 hours at room temperature. The reaction mixture was diluted with ethyl acetate and the ethyl acetate solution was washed with water. The organic layer was dried over anhydrous sodium sulfate. After filtering off the desiccant, the solvent was evaporated under reduced pressure. The resulting residue was purified by column chromatography (NH silica gel cartridge, hexane:ethyl acetate=90:10 to 20:80) to give the titled compound (2.00 g).

1H NMR (600 MHz, CHLOROFORM-d) d ppm 1.48-1.95 (m, 8H) 3.62-3.75 (m, 3H) 4.42-5.85 (m, 3H) 6.97-7.21 (m, 2H) 7.27-7.47 (m, 2H) 7.53 (s, 1H)

(ESI pos.) m/z: 380, 382 ([M+H]⁺)

The following compounds were synthesized by the same procedure

### N-(3-Bromo-4-fluorobenzyl)-1-methyl-N-(2-methylpropyl)-1H-imidazole-4-carboxamide

1H NMR (600 MHz, CHLOROFORM-d) d ppm 0.77-1.00 (m, 6H) 1.91-2.14 (m, 1H) 3.17-3.28 (m, 1H) 3.87-5.45 (m, 3H) 7.00-7.26 (m, 2H) 7.30-7.62 (m, 3H)

(ESI pos.) m/z: 368, 370 ([M+H]⁺)

### N-(3-Bromo-4-fluorobenzyl)-N-(propan-2-yl)-1H-1,2,4-triazole-3-carboxamide

1H NMR (600 MHz, CHLOROFORM-d) d ppm 1.17-1.29 (m, 6H) 4.65 (s, 3H) 7.01-7.25 (m, 2H) 7.42-8.60 (m, 2H)

(ESI pos.) m/z: 339, 341 ([M−H]⁻)

### N-(3-Bromo-4-fluorobenzyl)-N-cyclohexyl-1H-1,2,4-triazole-3-carboxamide

(ESI pos.) m/z: 379, 381 ([M−H]⁻)

### N-(3-Bromo-4-fluorobenzyl)-1-methyl-N-(propan-2-yl)-1H-imidazole-4-carboxamide

1H NMR (600 MHz, CHLOROFORM-d) d ppm 1.11-1.26 (m, 6H) 3.63-3.78 (m, 3 H) 4.48-5.79 (m, 3H) 6.95-7.59 (m, 5H)

(ESI pos.) m/z: 354, 356 ([M+H]⁺)

### N-(3-Bromo-4-fluorobenzyl)-N-cyclohexyl-1-methyl-1H-imidazole-4-carboxamide

1H NMR (600 MHz, CHLOROFORM-d) d ppm 0.99-1.86 (m, 10H) 3.70 (br. s., 3H) 4.33-5.32 (m, 3H) 6.95-7.58 (m, 5H)

(ESI pos.) m/z: 394, 396 ([M+H]⁺)

### N-(3-Bromo-4-fluorobenzyl)-N-(2-methoxyethyl)-1-methyl-1H-imidazole-4-carboxamide

1H NMR (600 MHz, CHLOROFORM-d) d ppm 3.25-3.34 (m, 3H) 3.48-3.75 (m, 6 H) 4.07-4.18 (m, 1H) 4.70-4.83 (m, 1H) 5.36-5.53 (m, 1H) 7.00-7.24 (m, 2H) 7.29-7.63 (m, 3H)

(ESI pos.) m/z: 370, 372 ([M+H]⁺)

### Preparation Example 5

### N-(3-Bromo-4-fluorobenzyl)-1-methyl-N-(propan-2-yl)-1H-1,2,4-triazole-3-carboxamide

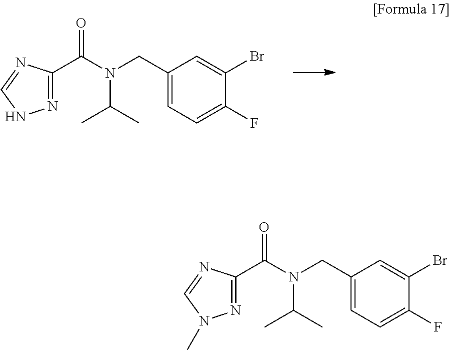

Sodium hydride (about 60% in oil, 266 mg) was added to a mixtue of N-(3-bromo-4-fluorobenzyl)-N-(propan-2-yl)-1H-1,2,4-triazole-3-carboxamide (2.06 g) and dimethylformamide (20 mL), and stirred for 30 minutes. After addition of methyl iodide (1.1 mL), the mixture was stirred overnight at room temperature. The reaction mixture was diluted with water and extracted with chloroform. The organic layer was dried over anhydrous sodium sulfate. After filtering off the desiccant, the organic layer was concentrated under reduced pressure. The resulting residue was purified by column chromatography (NH silica gel cartridge, hexane:ethyl acetate=50:50 to 34:66 to 0:100) to give the titled compound (1.00 g).

1H NMR (600 MHz, CHLOROFORM-d) d ppm 1.13-1.23 (m, 6H) 3.88-4.04 (m, 3 H) 4.60-4.83 (m, 3H) 7.00-7.08 (m, 1H) 7.20-8.13 (m, 3H)

(ESI pos.) m/z: 395, 397 ([M+H]⁺)

The following compound was synthesized by the same procedure.

### N-(3-Bromo-4-fluorobenzyl)-N-cyclohexyl-1-methyl-1H-1,2,4-triazole-3-carboxamide

1H NMR (600 MHz, CHLOROFORM-d) d ppm 0.95-1.89 (m, 10H) 3.83-4.85 (m, 6H) 6.95-8.17 (m, 4H)

(ESI pos.) m/z: 355, 357 ([M+H]⁺)

### Preparation Example 6

### N-(3-Bromo-4-fluorobenzyl)-1-methyl-1H-imidazole-4-carboxamide

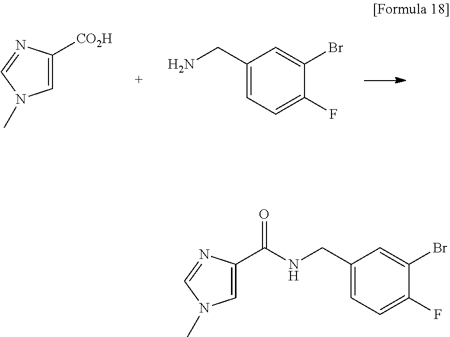

A mixture of 3-Bromo-4-fluorobenzylamine (1.62 g), 1-methyl-1H-imidazole-4-carboxylic acid (1.0 g), HATU (4.52 g), diisopropylethylamine (4.1 mL) and acetonitrile (26 ml) was stirred for 1 hour at room temperature. After addition of water and chloroform, the reaction mixture was stirred, and the chloroform layer was then separated and evaporated under reduced pressure. The resulting residue was purified by column chromatography (NH silica gel cartridge, hexane:ethyl acetate=50:50) and (silica gel cartridge, chloroform:methanol=99:1 to 90:10) to give the titled compound (1.87 g).

1H NMR (600 MHz, CHLOROFORM-d) d ppm 3.74 (s, 3H) 4.52-4.58 (m, 2H) 7.03-7.09 (m, 1H) 7.34-7.56 (m, 4H)

(ESI pos.) m/z: 312, 314 ([M+H]⁺)

### Preparation Example 7

### N-(3-Bromo-4-fluorobenzyl)-1-methyl-N-propyl-1H-imidazole-4-carboxamide

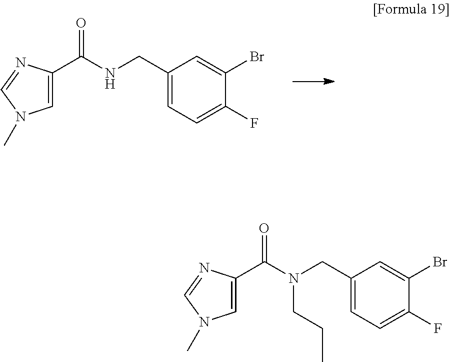

Sodium hydride (about 60% in oil, 24 mg) was added to a solution of N-(3-bromo-4-fluorobenzyl)-1-methyl-1H-imidazole-4-carboxamide (156 mg) in tetrahydrofuran (3 mL) and stirred for 30 minutes. Modopropane (136 mg) was added to the mixture and stirred for 4 hours at room temperature. Sodium hydride (about 60% in oil, 12 mg) was further added and stirred overnight. The reaction mixture was diluted with water and extracted with ethyl acetate. The organic layer was dried over anhydrous sodium sulfate. After filtering off the desiccant, the solvent was evaporated under reduced pressure. The resulting residue was purified by column chromatography (NH silica gel cartridge, hexane:ethyl acetate=90:10 to 20:80) to give the titled compound (56 mg).

1H NMR (600 MHz, CHLOROFORM-d) d ppm 0.81-0.94 (m, 3H) 1.59-1.69 (m, 2 H) 3.72 (s, 5H) 4.62-5.38 (m, 2H) 7.16-7.62 (m, 5H)

(ESI pos.) m/z: 354, 356 ([M+H]⁺)

### Preparation Example 8

### N-(4-Bromo-3-fluorobenzyl)propan-2-amine hydrochloride

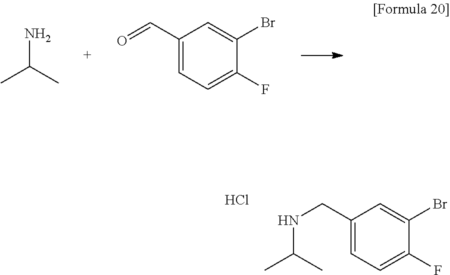

A mixture of isopropylamine (13.3 g), 3-bromo-4-fluorobenzaldehyde (13.3 g) and chloroform (300 mL) was stirred at room temperature for 30 minutes. Sodium triacetoxyborohydride (47.7 g) was added to the mixture in several portions and stirred at room temperature for 15 hours. 2 M aqueous sodium hydroxide (150 mL) was added and stirred at room temperature for 30 minutes. The chloroform layer was separated and the aqueous layer was extracted with chloroform. The combined chloroform layers were washed with water and dried over anhydrous sodium sulfate. After filtering off the desiccant, chloroform was evaporated under reduced pressure. The resulting residue was dissolved in ethyl acetate (150 mL), followed by addition of 4 M hydrochloric acid/ethyl acetate (150 mL). The precipitation was collected by filtration to give the titled compound (29.0 g).

1H NMR (600 MHz, DMSO-d6) d ppm 1.29 (d, J=6.42 Hz, 6H) 3.22-3.34 (m, 1H) 4.13 (s, 2H) 7.42-7.51 (m, 1H) 7.60-7.68 (m, 1H) 7.95-8.04 (m, 1H) 8.95-9.24 (m, 2H)

The following compounds were synthesized by the same procedure.

### N-(3-Bromo-4-methoxybenzyl)propan-2-amine hydrochloride

1H NMR (200 MHz, DMSO-d6) d ppm 1.20-1.37 (m, 6H) 3.14-3.37 (m, 1H) 3.87 (s, 3H) 3.98-4.17 (m, 2H) 7.18 (d, J=8.35 Hz, 1H) 7.55 (d, J=8.35 Hz, 1H) 7.82 (s, 1H) 8.98 (br. s., 2H)

(ESI pos.) m/z: 258, 260 ([M+H]⁺)

### N-(3-Bromo-5-chlorobenzyl)propan-2-amine hydrochloride

1H NMR (200 MHz, DMSO-d6) d ppm 1.23-1.35 (m, 6H) 3.17-3.40 (m, 1H) 4.05-4.23 (m, 2H) 7.71-7.97 (m, 3H) 9.19 (br. s., 2H)

(ESI pos.) m/z: 262, 264 ([M+H]⁺)

### N-(3-Bromo-4-methylbenzyl)propan-2-amine hydrochloride

1H NMR (600 MHz, DMSO-d6) d ppm 1.24-1.31 (m, 6H) 2.35 (s, 3H) 3.22-3.30 (m, 1H) 4.10 (s, 2H) 7.40-7.43 (m, 1H) 7.46-7.49 (m, 1H) 7.83 (s, 1H) 8.97 (br. s., 1H)

(ESI pos.) m/z: 242, 244 ([M+H]⁺)

### N-[3-Bromo-5-(trifluoromethyl)benzyl]propan-2-amine hydrochloride

1H NMR (600 MHz, DMSO-d6) d ppm 1.30 (d, J=6.88 Hz, 6H) 4.25 (s, 2H) 8.01-8.20 (m, 3H) 9.04-9.22 (m, 2H)

(ESI pos.) m/z: 296, 298 ([M+H]⁺)

### N-[3-Bromo-5-(trifluoromethoxy)benzyl]propan-2-amine hydrochloride

1H NMR (200 MHz, DMSO-d6) d ppm 1.30 (d, J=6.59 Hz, 6H) 3.20-3.40 (m, 1H) 4.13-4.30 (m, 2H) 7.72 (s, 1H) 7.75 (s, 1H) 7.94 (s, 1H) 9.22 (br. s., 2H)

(ESI pos.) m/z: 312, 314 ([M+H]⁺)

### N-(3-Bromo-4-chlorobenzyl)propan-2-amine hydrochloride

1H NMR (200 MHz, DMSO-d6) d ppm 1.29 (d, J=6.59 Hz, 6H) 3.10-3.50 (m, 1H) 4.14 (s, 1H) 7.58-7.68 (m, 1H) 7.68-7.77 (m, 1H) 8.02-8.12 (m, 1H) 9.23 (br. s., 2H)

(ESI pos.) m/z: 262, 264 ([M+H]⁺)

### Preparation Example 9

### N-(3-Bromo-4-fluorobenzyl)-N-(propan-2-yl)-1H-imidazole-4-carboxamide

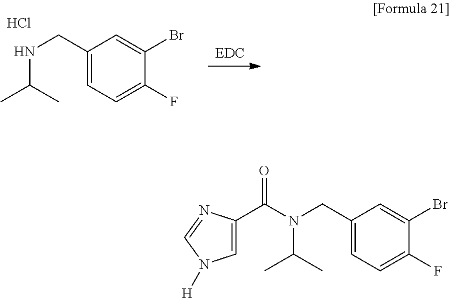

A mixture of N-(4-bromo-3-fluorobenzyl)propan-2-amine hydrochloride (2.3 g), 1H-imidazole-4-carboxylic acid (1.18 g), HOBT (1.61 g), EDC hydrochloride (2.68 g), triethylamine (2.4 mL) and dimethylformamide (40 mL) was stirred at room temperature for 6 days. The reaction mixture was concentrated under reduced pressure, and the residue was diluted with saturated aqueous sodium bicarbonate and stirred for 30 minutes. The resulting mixture was extracted with ethyl acetate, and the ethyl acetate layer was washed with water. The ethyl acetate layer was dried over anhydrous sodium sulfate. After filtering off the desiccant, the solvent was evaporated under reduced pressure. The resulting residue was purified by column chromatography (NH silica gel cartridge, hexane/ethyl acetate=90:10 to 10:90) to give the titled compound (10.2 g).

1H NMR (600 MHz, CHLOROFORM-d) d ppm 1.04-1.37 (m, 6H) 4.49-5.78 (m, 3 H) 6.95-7.82 (m, 5H)

(ESI pos.) m/z: 340, 342 ([M+H]⁺)

The following compounds were synthesized by the same procedure.

### N-(3-Bromo-4-methoxybenzyl)-1-methyl-N-(propan-2-yl)-1H-imidazole-4-carboxamide

1H NMR (600 MHz, CHLOROFORM-d) d ppm 1.06-1.30 (m, 6H) 3.70 (s, 3H) 3.86 (s, 3H) 4.41-5.81 (m, 3H) 6.74-7.63 (m, 5H)

(ESI pos.) m/z: 366, 368 ([M+H]⁺)

### N-(3-Bromo-5-chlorobenzyl)-1-methyl-N-(propan-2-yl)-1H-imidazole-4-carboxamide

1H NMR (600 MHz, CHLOROFORM-d) d ppm 1.05-1.30 (m, 6H) 3.72 (s, 3H) 4.46-5.83 (m, 3H) 7.16-7.61 (m, 5H)

(ESI pos.) m/z: 370, 372 ([M+H]⁺)

### N-(3-Bromo-4-methylbenzyl)-1-methyl-N-(propan-2-yl)-1H-imidazole-4-carboxamide

1H NMR (600 MHz, CHLOROFORM-d) d ppm 1.10-1.25 (m, 6H) 3.63-3.79 (m, 3 H) 4.52-5.82 (m, 3H) 7.07-7.60 (m, 5H)

(ESI pos.) m/z: 350, 352 ([M+H]⁺)

### N-[3-Bromo-5-(trifluoromethyl)benzyl]-1-methyl-N-(propan-2-yl)-1H-imidazole-4-carboxamide

1H NMR (600 MHz, CHLOROFORM-d) d ppm 1.04-1.30 (m, 6H) 3.60-3.80 (m, 3 H) 4.56-5.91 (m, 3H) 7.16-7.70 (m, 5H)

(ESI pos.) m/z: 404, 406 ([M+H]⁺)

### N-[3-Bromo-5-(trifluoromethoxy)benzyl]-1-methyl-N-(propan-2-yl)-1H-imidazole-4-carboxamide

1H NMR (600 MHz, CHLOROFORM-d) d ppm 1.10-1.31 (m, 6H) 3.62-3.80 (m, 3 H) 4.50-5.87 (m, 3H) 7.04-7.63 (m, 5H)

(ESI pos.) m/z: 420, 422 ([M+H]⁺)

### N-(3-Bromo-4-chlorobenzyl)-1-methyl-N-(propan-2-yl)-1H-imidazole-4-carboxamide

1H NMR (600 MHz, CHLOROFORM-d) d ppm 1.03-1.32 (m, 6H) 3.71 (s, 3H) 4.41-5.86 (m, 3H) 7.10-7.62 (m, 5H)

(ESI pos.) m/z: 370, 372 ([M+H]⁺)

### Preparation Example 10

### N-[(3′,4′,5′,6-Tetrafluorobiphenyl-3-yl)methyl]propan-2-amine

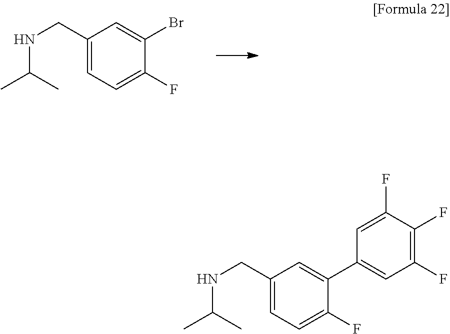

A mixture of N-(4-bromo-3-fluorobenzyl)propan-2-amine (500 mg), (3,4,5-trifluorophenyl)boronic acid (386 mg), cesium carbonate (780 mg), tetrakis(triphenylphosphine) palladium (230 mg), toluene (2.2 mL), ethanol (2.2 mL) and water (1.4 mL) was reacted in a microwave reaction apparatus at 150° C. for 30 minutes. After cooling, the reaction mixture was diluted with saturated sodium bicarbonate and extracted with ethyl acetate. The ethyl acetate layer was dried over anhydrous sodium sulfate. After filtering off the desiccant, the solvent was evaporated under reduced pressure, and the resulting residue was purified by chromatography (silica gel cartridge, chloroform/methanol=100:0 to 90:10) to give the titled compound (628 mg).

1H NMR (600 MHz, CHLOROFORM-d) d ppm 1.11 (d, J=6.30 Hz, 6H) 2.87 (spt, J=6.30 Hz, 1H) 3.79 (s, 2 μl) 7.08-7.14 (m, 1H) 7.16-7.22 (m, 2H) 7.30-7.36 (m, 2H)

(ESI pos.) m/z: 298 ([M+H]⁺)

The following compounds were synthesized by the same procedure. 2-({[6-Fluoro-4′-(trifluoromethoxy)biphenyl-3-yl]methyl}amino)propan-1-ol

1H NMR (600 MHz, CHLOROFORM-d) d ppm 1.10-1.13 (m, 4H) 2.85-2.91 (m, 1 H) 3.28-3.34 (m, 1H) 3.60-3.65 (m, 1H) 3.75-3.80 (m, 1H) 3.89-3.94 (m, 1H) 7.09-7.15 (m, 1H) 7.27-7.41 (m, 4H) 7.54-7.59 (m, 2H)

(ESI pos.) m/z: 344 ([M+H]⁺)

### 1-[6-Fluoro-4′-(trifluoromethoxy)biphenyl-3-yl]methanamine

1H NMR (200 MHz, CHLOROFORM-d) d ppm 3.91 (s, 2H) 7.05-7.64 (m, 7H)

### Example 1

### N-{[6-Fluoro-4′-(trifluoromethoxy)biphenyl-3-yl]methyl}-1-methyl-N-(propan-2-yl)-1H-imidazole-4-carboxamide

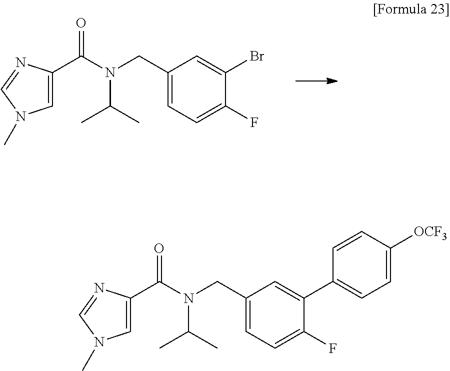

A mixture of N-(3-bromo-4-fluorobenzyl)-1-methyl-N-(propan-2-yl)-1H-imidazole-4-carboxamide (127 mg), 4-trifluoromethoxyphenylboronic acid (154 mg), tetrakis(triphenylphosphine)palladium (45 mg), potassium carbonate (70 mg), dimethylformamide (3 mL) and ethanol (1.5 mL) was reacted in a microwave reaction apparatus (150° C., 25 minutes). After addition of ethyl acetate and filtration through Celite® pad, the ethyl acetate solution was washed with water. The ethyl acetate layer was dried over anhydrus sodium sulfate. After filtering off the desiccant, the ethyl acetate layer was concentrated under reduced pressure. The resulting residue was purified by column chromatography (NH silica gel cartridge, hexane:ethyl acetate=90:10 to ethyl acetate) and (silica gel cartridge, chloroform:methanol=100:0 to 97:3) to give the titled compound (77 mg).

### Example 2

### N-{[6-Fluoro-4′-(trifluoromethoxy)biphenyl-3-yl]methyl}-1-methyl-N-propyl-1H-imidazole-4-carboxamide hydrochloride

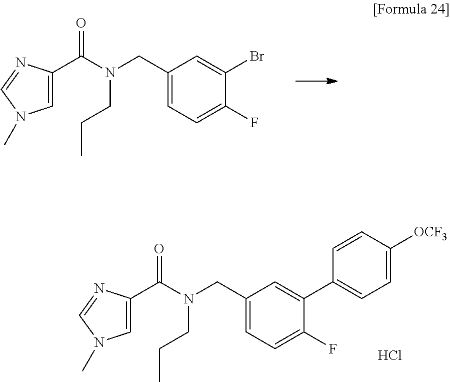

A mixture of N-(3-bromo-4-fluorobenzyl)-1-methyl-N-propyl-1H-imidazole-4-carboxamide (56 mg), 4-trifluoromethoxyphenylboronic acid (65 mg), tetrakis(triphenylphosphine)palladium (20 mg), potassium carbonate (44 mg), dimethylformamide (1.3 mL) and ethanol (0.6 mL) was reacted in a microwave reaction apparatus (150° C., 25 minutes). After addition of ethyl acetate, the ethyl acetate solution was washed with water. The ethyl acetate layer was dried over anhydrus sodium sulfate. After filtering off the desiccant, the ethyl acetate layer was concentrated under reduced pressure. The resulting residue was purified by column chromatography (NH silica gel cartridge, hexane:ethyl acetate=80:20 to 20:80) and (silica gel cartridge, chloroform:methanol=100:0 to 97:3). The resulting residue was dissolved in ethyl acetate, and 4 N hydrochloric acid/ethyl acetate (about 1 mL) was added thereto and stirred for 5 minutes, followed by distilling off the solvent under reduced pressure. The residue was solidified by addition of diethyl ether, and the solid was collected by filtration to give the titled compound (24 mg).

### Example 3

### Methyl 2′-fluoro-5′-({[(1-methyl-1H-1,2,4-triazol-3-yl)carbonyl](propan-2-yl)amino}methyl)biphenyl-3-carboxylate

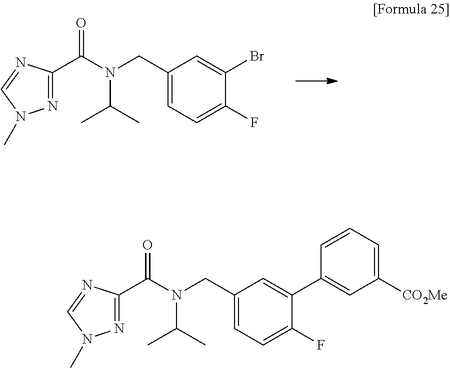

A mixture of N-(3-bromo-4-fluorobenzyl)-1-methyl-N-(propan-2-yl)-1H-1,2,4-triazole-3-carboxamide (492 mg), 3-methoxycarbonylphenylboronic acid (274 mg), tetrakis(triphenylphosphine)palladium (161 mg), cesium carbonate (679 mg) and toluene/ethanol/water (3:3:2, 9 mL) was stirred at 100° C. for 1 hour. The reaction mixture was diluted with water and extracted with ethyl acetate. The organic layer was dried over anhydrous sodium sulfate. After filtering off the desiccant, the organic layer was concentrated under reduced pressure. The resulting residue was purified by column chromatography (NH silica gel cartridge, hexane:ethyl acetate=50:50 to 0:100) to give the titled compound (486 mg).

### Example 4

### N-{([6-Fluoro-3′-(hydroxymethyl)biphenyl-3-yl]methyl}-1-methyl-N-(propan-2-yl)-1H-1,2,4-triazole-3-carboxamide

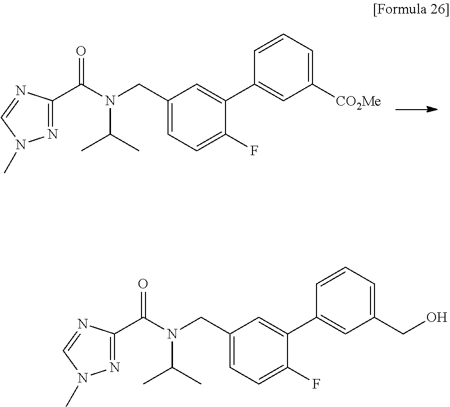

Sodium borohydride (723 mg) was added to a mixture of methyl 2′-fluoro-5′-({[(1-methyl-1H-1,2,4-triazol-3-yl)carbonyl](propan-2-yl)amino}methyl)biphenyl-3-carboxylate (392 mg) in ethanol (19 mL), and refluxed overnight. Sodium borohydride (362 mg) was further added and refluxed for 5 hours. After cooling, acetone was added to the reaction mixture and stirred, and then diluted with water and extracted with chloroform. The organic layer was dried over anhydrous sodium sulfate. After filtering off the desiccant, the organic layer was concentrated under reduced pressure. The resulting residue was purified by column chromatography (silica gel cartridge, chloroform/methanol=99:1 to 97:3) to give the titled compound (180 mg).

### Example 5

### N-Cyclobutyl-N-{[6-fluoro-3′-(hydroxymethyl)biphenyl-3-yl]methyl}-1-methyl-1H-imidazole-4-carboxamide

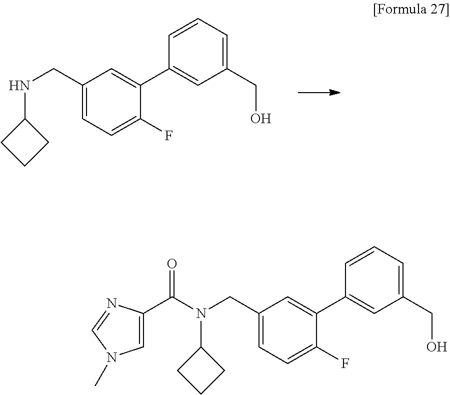

A mixture of {5′-[(cyclobutylamino)methyl]-2′-fluorobiphenyl-3-yl}methanol (700 mg), 1-methyl-1H-imidazole-4-carboxylic acid (340 mg), HATU (1.21 g), diisopropylethylamine (1.13 mL) and acetonitrile (10.5 ml) was stirred for 2.5 hours at room temperature. The reaction mixture was diluted with ethyl acetate and the organic layer was washed with water. The organic layer was dried over anhydrous sodium sulfate. After filtering off the desiccant, the solvent was evaporated under reduced pressure. The resulting residue was purified by column chromatography (silica gel cartridge, chloroform:methanol=98:2 to 95:5) and (NH silica gel cartridge, chloroform:methanol=100:0 to 98:2) to give the titled compound (758 mg).

### Example 6

### 1-Ethyl-N-{[6-fluoro-4′-(trifluoromethoxy)biphenyl-3-yl]methyl}-N-(propan-2-yl)-1H-imidazole-4-carboxamide

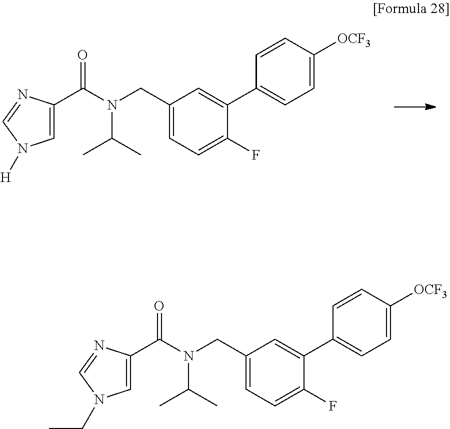

Sodium hydride (about 60% in oil, 12 mg) was added to a solution of N-{[6-fluoro-4′-(trifluoromethoxy)biphenyl-3-yl]methyl}-N-(propan-2-yl)-1H-imidazole-4-carboxamide (120 mg) in dimethylformamide (2 mL) and stirred at room temperature for 15 minutes. Iodoethane (134 mg) was added to the mixture and stirred at 80° C. for 2 hours. Saturated aqueous sodium bicarbonate was added to the reaction mixture, which was then extracted with ethyl acetate. The organic layer was washed with brine and concentrated under reduced pressure, and the resulting residue was purified by preparative HPLC to give the titled compound (62 mg).

### Example 7

### N-{[6-Fluoro-4′-(trifluoromethoxy)biphenyl-3-yl]methyl}-1-methyl-N-(prop-1-en-2-yl)-1H-imidazole-4-carboxamide

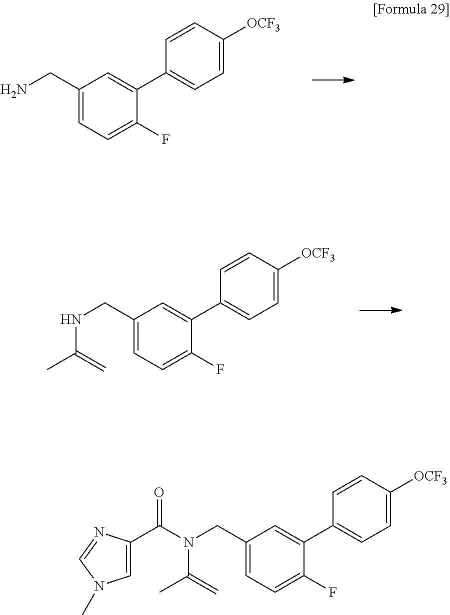

A mixture of 1-[6-fluoro-4′-(trifluoromethoxy)biphenyl-3-yl]methanamine (500 mg) and acetone (1 mL) was reacted in a microwave reaction apparatus at 120° C. for 1 hour. Anhydrous sodium sulfate was added to the reaction mixture. After filtering off the desiccant, the solvent was evaporated under reduced pressure to give N-{[6-fluoro-4′-(trifluoromethoxy)biphenyl-3-yl]methyl}prop-1-en-2-amine.

Chloroform (10 mL), triethylamine (265 mg) and 1-methyl-1H-imidazole-4-carboxylic acid chloride (260 mg) were added to the obtained N-{[6-fluoro-4′-(trifluoromethoxy)biphenyl-3-yl]methyl}prop-1-en-2-amine and stirred at room temperature for 2 days. The reaction mixture was diluted with water and extracted with chloroform. After the chloroform layer was separated with a phase separator, the solvent was evaporated under reduced pressure, and the resulting residue was purified by HPLC and TLC to give the titled compound (30 mg).

Tables 1-1 to 1-6 show the structural formulae of the compounds shown in Examples 1 to 7 and compounds synthesized in the same manner with their analytical data. Each numeral in the column “Example” in each table represents Example No. corresponding to the synthetic procedure of the intended compound, i.e., it means that the intended compound was synthesized in the same manner as shown in the indicated Example No.

TABLE 1-1

| | | | | | (ESI pos.) |
|---|---|---|---|---|---|
| | Ex- | | | | m/z |
| Com- | am- | | | | (ESI neg.) |
| pound | ple | Structure | Salt | NMR | m/z |
| 1 | 3 | 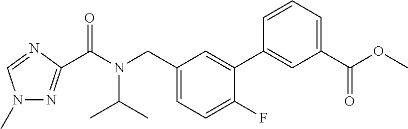 | | 1H NMR (600 MHz, CHLOROFORM-d) d ppm 1.13-1.31 (m, 6 H) 3.87-4.03 (m, 6 H) 4.59-4.91 (m, 3 H) 7.06-8.25 (m, 8 H) | 411 ([M + H]+) |
| 2 | 3 | 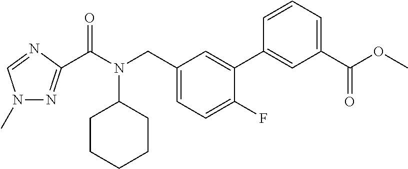 | | 1H NMR (600 MHz, CHLOROFORM-d) d ppm 0.99-1.88 (m, 10 H) 3.86-4.01 (m, 6H) 4.39-4.86 (m, 3 H) 6.91-7.11 (m, 1 H) 7.28-8.22 (m, 7 H) | 451 ([M + H]+) |
| 3 | 1 | 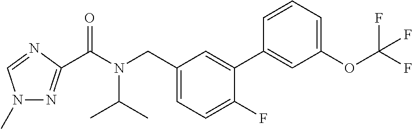 | | 1H NMR (600 MHz, CHLOROFORM-d) d ppm 1.16-1.27 (m, 6 H) 3.87-4.03 (m, 3 H) 4.62-4.89 (m, 3 H) 7.05-8.11 (m, 8 H) | 437 ([M + H]+) |
| 4 | 1 | 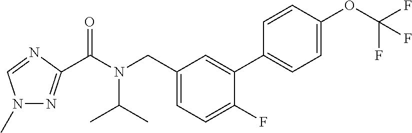 | | 1H NMR (600 MHz, CHLOROFORM-d) d ppm 1.18-1.25 (m, 6 H) 3.88-4.01 (m, 3 H) 4.62-4.88 (m, 3 H) 7.05-8.11 (m, 8 H) | 437 ([M + H]+) |
| 5 | 4 | 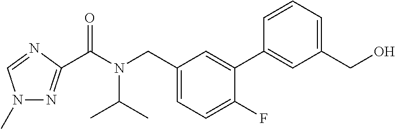 | | 1H NMR (600 MHz, CHLOROFORM-d) d ppm 1.14-1.24 (m, 6 H) 3.86-4.02 (m, 3 H) 4.58-4.90 (m, 5 H) 7.04-7.12 (m, 1 H) 7.30- 8.10 (m, 7 H) | 383 ([M + H]+) |
| 6 | 4 | 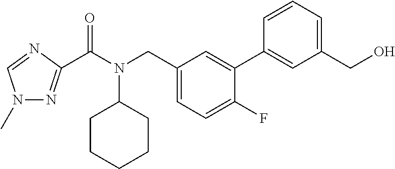 | | 1H NMR (600 MHz, CHLOROFORM-d) d ppm 0.95-1.89 (m, 10 H) 3.85-4.88 (m, 8H) 7.03-8.11 (m, 8 H) | 423 ([M + H]+) |
| 7 | 5 | 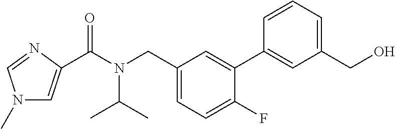 | | 1H NMR (600 MHz, CHLOROFORM-d) d ppm 1.14-1.25 (m, 6 H) 3.61-3.76 (m, 3 H) 4.57-5.76 (m, 5 H) 6.99-7.65 (m, 9 H) | 382 ([M + H]+) |
| 8 | 5 | 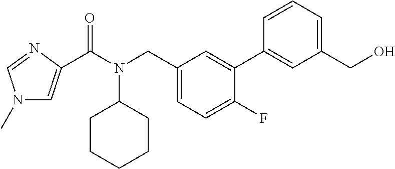 | | 1H NMR (600 MHz, CHLOROFORM-d) d ppm 0.96-1.92 (m, 10 H) 3.59-3.79 (m, 3H) 4.32-5.40 (m, 5 H) 6.98-7.59 (m, 9 H) | 422 ([M + H]+) |
| 9 | 1 | 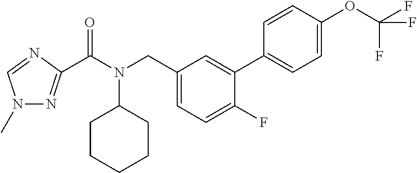 | | 1H NMR (600 MHz, CHLOROFORM-d) d ppm 0.97-1.87 (m, 10H) 3.87-4.87 (m, 6H) 7.03-8.11 (m, 8H) | 477 ([M + H]+) |
| 10 | 1 | 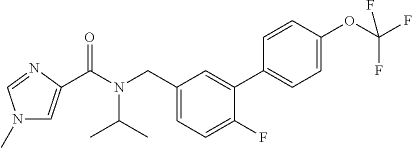 | | 1H NMR (600 MHz, CHLOROFORM-d) d ppm 1.07-1.33 (m, 6 H) 3.71 (br. s., 3 H) 4.55-5.85 (m, 3 H) 7.01-7.13 (m, 1 H) 7.18- 7.60 (m, 8 H) | 436 ([M + H]+) |

TABLE 1-2

| | | | | | (ESI pos.) |
|---|---|---|---|---|---|
| | Ex- | | | | m/z |
| Com- | am- | | | | (ESI neg.) |
| pound | ple | Structure | Salt | NMR | m/z |
| 11 | 5 | 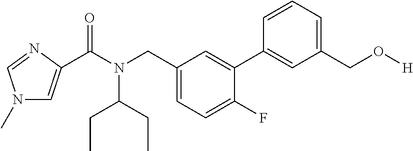 | | 1H NMR (600 MHz, CHLOROFORM-d) d ppm 0.80-0.90 (m, 6 H) 1.57-1.80 (m, 5 H) 3.66-3.74 (m, 3 H) 4.55-5.39 (m, 5 H) 7.00- 7.08 (m, 1 H) 7.28-7.60 (m, 8 H) | 410 ([M + H]+) 408 ([M − H]−) |
| 12 | 2 | 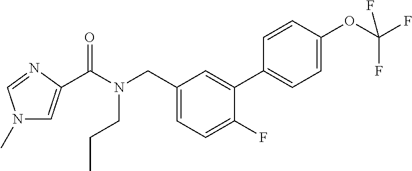 | HCl | 1H NMR (600 MHz, METHANOL-d3) d ppm 0.87-0.99 (m, 3 H) 1.64-1.81 (m, 2 H) 3.39-4.08 (m, 5 H) 4.88-5.00 (m, 1 H) 7.14-8.02 (m, 8 H) 8.97 (br. s., 1 H) | 436 ([M + H]+) |
| 13 | 5 | 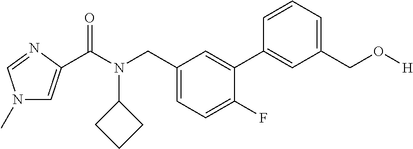 | | 1H NMR (600 MHz, CHLOROFORM-d) d ppm 1.61-1.79 (m, 2 H) 2.04-2.24 (m, 4 H) 3.61-3.79 (m, 3 H) 4.50-5.80 (m, 5 H) 6.99- 7.60 (m, 9 H) | 394 ([M + H]+) |
| 14 | 5 | 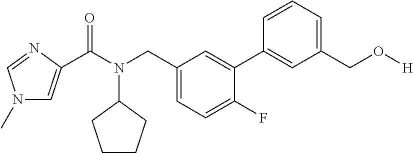 | | 1H NMR (600 MHz, CHLOROFORM-d) d ppm 1.44-2.00 (m, 8 H) 3.57-3.77 (m, 3 H) 4.50-5.80 (m, 5 H) 6.98-7.55 (m, 9 H) | 408 ([M + H]+) |
| 15 | 1 | 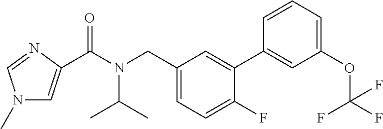 | | 1H NMR (600 MHz, CHLOROFORM-d) d ppm 1.15-1.26 (m, 6 H) 3.66-3.76 (m, 3 H) 4.58-5.79 (m, 3 H) 7.04-7.61 (m, 9 H) | 436 ([M + H]+) |
| 16 | 1 | 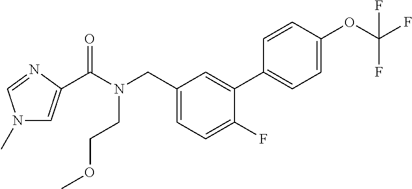 | | 1H NMR (600 MHz, CHLOROFORM-d) d ppm 3.27-3.36 (m, 3H) 3.54-3.75 (m, 6H) 4.13-4.21 (m, 1H) 4.80-4.89 (m, 1H) 5.45- 5.56 (m, 1H) 7.04-7.14 (m, 1H) 7.61 (s, 8H) | 452 ([M + H]+) |
| 17 | 1 | 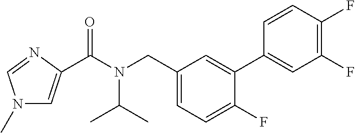 | | 1H NMR (600 MHz, CHLOROFORM-d) d ppm 1.11-1.30 (m, 6 H) 3.61-3.78 (m, 3 H) 4.56-5.81 (m, 3 H) 6.99-7.65 (m, 8 H) | 388 ([M + H]+) |
| 18 | 1 | 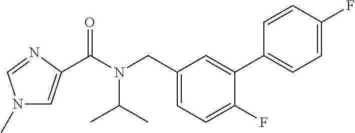 | | 1H NMR (600 MHz, CHLOROFORM-d) d ppm 1.11-1.31 (m, 6 H) 3.60-3.77 (m, 3 H) 4.52-5.84 (m, 3 H) 7.01-7.59 (m, 9 H) | 370 ([M + H]+) |
| 19 | 1 | 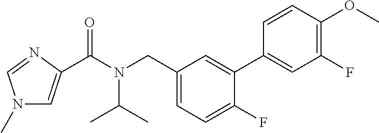 | | 1H NMR (600 MHz, CHLOROFORM-d) d ppm 1.12-1.29 (m, 6 H) 3.63-3.77 (m, 3 H) 4.58-5.80 (m, 3 H) 6.97-7.60 (m, 8 H) | 400 ([M + H]+) |
| 20 | 2 | 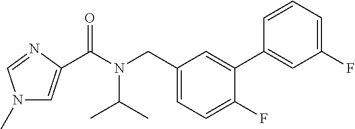 | HCl | 1H NMR (600 MHz, METHANOL-d3) d ppm 1.24-1.39 (m, 6 H) 3.79-4.07 (m, 3 H) 4.57-4.68 (m, 1 H) 7.08-8.16 (m, 8 H) 8.97-9.05 (m, 1 H) | 370 ([M + H]+) |

TABLE 1-3

| | | | | | (ESI pos.) |
|---|---|---|---|---|---|
| | Ex- | | | | m/z |
| Com- | am- | | | | (ESI neg.) |
| pound | ple | Structure | Salt | NMR | m/z |
| 21 | 2 | 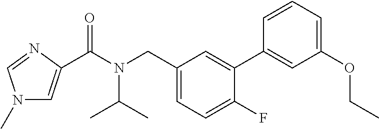 | HCl | 1H NMR (600 MHz, METHANOL-d3) d ppm 1.26-1.47 (m, 6 H) 3.80-4.14 (m, 3 H) 4.56-4.69 (m, 1 H) 6.90-8.14 (m, 8 H) 8.91-9.02 (m, 1 H) | 396 ([M + H]+) |
| 22 | 1 | 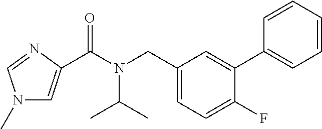 | | 1H NMR (600 MHz, CHLOROFORM-d) d ppm 1.11-1.31 (m, 6 H) 3.60-3.77 (m, 3 H) 4.57-5.82 (m, 3 H) 7.00-7.60 (m, 10 H) | 352 ([M + H]+) |
| 23 | 1 | 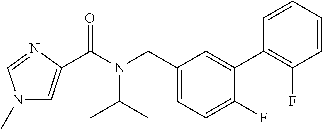 | | 1H NMR (600 MHz, CHLOROFORM-d) d ppm 1.12-1.28 (m, 6 H) 3.60-3.79 (m, 3 H) 4.52-5.79 (m, 3 H) 7.02-7.58 (m, 9 H) | 370 ([M + H]+) |
| 24 | 2 | 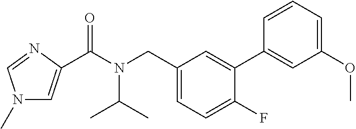 | HCl | 1H NMR (600 MHz, METHANOL-d3) d ppm 1.30 (d, J = 6.42 Hz, 6 H) 3.82 (s, 3 H) 3.86-4.04 (m, 2 H) 4.59-4.68 (m, 1 H) 6.93-8.09 (m, 8 H) 8.88-8.97 (m, 1 H) | 382 ([M + H]+) |
| 25 | 1 | 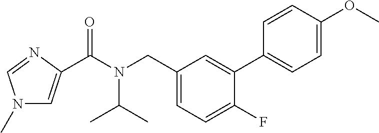 | | 1H NMR (600 MHz, CHLOROFORM-d) d ppm 1.14-1.27 (m, 6 H) 3.63-3.75 (m, 3 H) 3.84 (s, 3 H) 4.57-5.79 (m, 3 H) 6.92-7.57 (m, 9 H) | 382 ([M + H]+) |
| 26 | 2 | 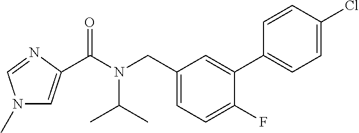 | HCl | 1H NMR (600 MHz, METHANOL-d3) d ppm 1.23-1.40 (m, 6 H) 3.76-4.06 (m, 3 H) 4.56-4.83 (m, 3 H) 7.10-8.17 (m, 8 H) 8.91-9.04 (m, 1 H) | 386 ([M + H]+) |
| 27 | 2 | 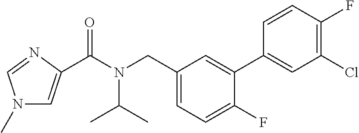 | HCl | 1H NMR (600 MHz, METHANOL-d3) d ppm 1.23-1.41 (m, 6 H) 3.79-4.05 (m, 3 H) 4.56-4.82 (m, 3 H) 7.12-8.19 (m, 7 H) 8.88-9.06 (m, 1 H) | 404 ([M + H]+) |
| 28 | 2 | 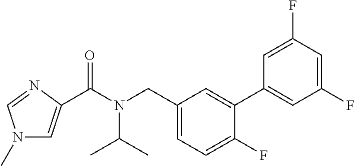 | HCl | 1H NMR (600 MHz, METHANOL-d3) d ppm 1.30 (d, J = 4.13 Hz, 6 H) 3.79- 4.06 (m, 3 H) 4.55-4.69 (m, 1 H) 6.94- 8.15 (m, 7 H) 8.93-9.06 (m, 1 H) | 388 ([M + H]+) |
| 29 | 2 | 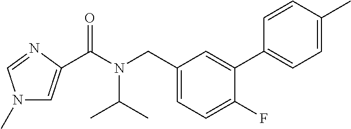 | HCl | 1H NMR (600 MHz, METHANOL-d3) d ppm 1.25-1.36 (m, 6 H) 2.38 (s, 3 H) 3.77-4.04 (m, 3 H) 4.56-4.68 (m, 1 H) 7.07-8.14 (m, 7 H) 8.94-9.03 (m, 1 H) | 366 ([M + H]+) |
| 30 | 2 | 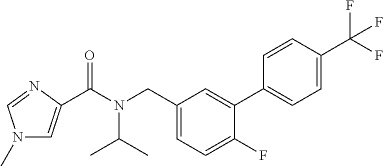 | HCl | 1H NMR (600 MHz, METHANOL-d3) d ppm 1.21-1.37 (m, 6 H) 3.77-4.00 (m, 3 H) 4.63-4.82 (m, 3 H) 7.12-8.04 (m, 8 H) 8.66-8.81 (m, 1 H) | 420 ([M + H]+) |

TABLE 1-4

| | | | | | (ESI pos.) |
|---|---|---|---|---|---|
| | Ex- | | | | m/z |
| Com- | am- | | | | (ESI neg.) |
| pound | ple | Structure | Salt | NMR | m/z |
| 31 | 2 | 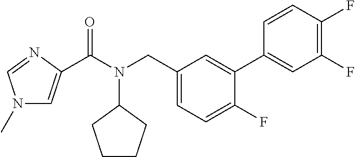 | HCl | 1H NMR (600 MHz, METHANOL-d3) d ppm 1.54-1.82 (m, 6 H) 1.89-2.01 (m, 2 H) 3.83-4.07 (m, 3 H) 4.61-4.81 (m, 3 H) 7.12-8.16 (m, 7 H) 8.88-9.07 (m, 1 H) | 414 ([M + H]+) |
| 32 | 2 | 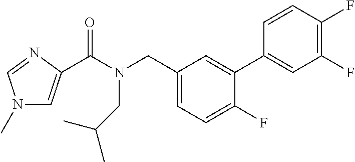 | HCl | 1H NMR (600 MHz, METHANOL-d3) d ppm 0.83-1.02 (m, 6 H) 2.02-2.22 (m, 1 H) 3.34-3.53 (m, 2 H) 3.82-4.03 (m, 3 H) 4.88-5.02 (m, 2 H) 7.14-8.09 (m, 7 H) 8.88-9.00 (m, 1 H) | 402 ([M + H]+) |
| 33 | 2 | 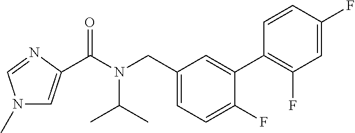 | HCl | 1H NMR (600 MHz, METHANOL-d3) d ppm 1.17-1.43 (m, 6 H) 3.79-4.07 (m, 3 H) 4.53-4.82 (m, 3 H) 7.00-8.16 (m, 7 H) 8.90-9.08 (m, 1 H) | 410 ([M + H]+) |
| 34 | 2 | 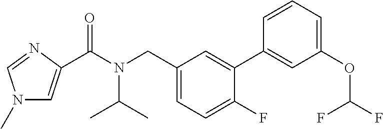 | HCl | 1H NMR (600 MHz, METHANOL-d3) d ppm 1.21-1.45 (m, 6 H) 3.76-4.10 (m, 3 H) 4.56-4.71 (m, 1 H) 6.67-9.09 (m, 10 H) | 418 ([M + H]+) |
| 35 | 2 | 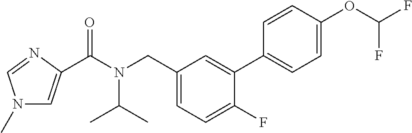 | HCl | 1H NMR (600 MHz, METHANOL-d3) d ppm 1.16-1.41 (m, 6 H) 3.75-4.10 (m, 3 H) 4.51-4.75 (m, 1 H) 6.68-9.06 (m, 10 H) | 418 ([M + H]+) |
| 36 | 2 | 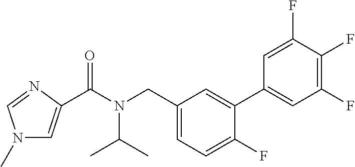 | HCl | 1H NMR (600 MHz, METHANOL-d3) d ppm 1.12-1.49 (m, 6 H) 3.77-4.16 (m, 3 H) 4.53-4.73 (m, 1 H) 7.10-9.12 (m, 7 H) | 406 ([M + H]+) |
| 37 | 1 | 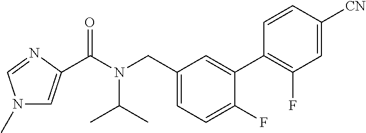 | | 1H NMR (600 MHz, CHLOROFORM-d) d ppm 1.09-1.35 (m, 6 H) 3.63-3.81 (m, 3 H) 4.55-5.79 (m, 3 H) 7.04-7.63 (m, 8 H) | 395 ([M + H]+) |
| 38 | 1 | 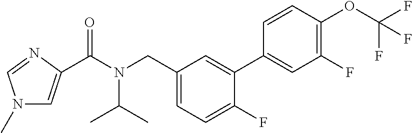 | | 1H NMR (600 MHz, CHLOROFORM-d) d ppm 1.06-1.38 (m, 6 H) 3.62-3.78 (m, 3 H) 4.53-5.84 (m, 3 H) 7.08 (dd, J = 10.55, 8.25 Hz, 1 H) 7.19-7.65 (m, 7 H) | 454 ([M + H]+) |
| 39 | 1 | 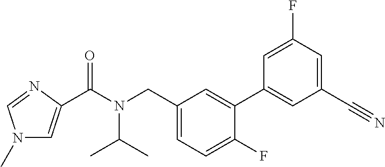 | | 1H NMR (600 MHz, CHLOROFORM-d) d ppm 1.17-1.30 (m, 6 H) 3.72 (s, 3 H) 4.56- 5.85 (m, 3 H) 7.05-7.16 (m, 1 H) 7.29-7.67 (m, 7 H) | 395 ([M + H]+) |

TABLE 1-5

| | | | | | (ESI pos.) |
|---|---|---|---|---|---|
| | Ex- | | | | m/z |
| Com- | am- | | | | (ESI neg.) |
| pound | ple | Structure | Salt | NMR | m/z |
| 40 | 6 | 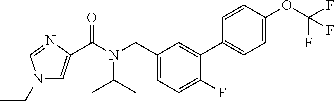 | | 1H NMR (600 MHz, CHLOROFORM-d) d ppm 1.14-1.30 (m, 6 H) 1.41-1.52 (m, 3 H) 3.93-4.07 (m, 2 H) 4.57-5.85 (m, 3 H) 7.03-7.10 (m, 1 H) 7.22-7.65 (m, 8 H) | 450 ([M + H]+) |
| 41 | 5 | 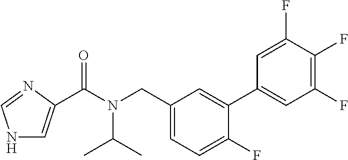 | | 1H NMR (600 MHz, CHLOROFORM-d) d ppm 1.17-1.35 (m, 6 H) 4.52-5.35 (m, 3 H) 7.07-7.17 (m, 3 H) 7.27-7.71 (m, 4 H) | 392 ([M + H]+) 390 ([M − H]−) |
| 42 | 6 | 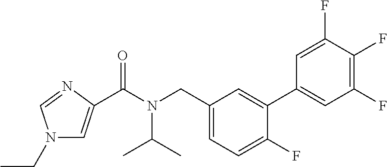 | | 1H NMR (600 MHz, DMSO-d6) d ppm 1.08-1.53 (m, 8 H) 4.00-4.31 (m, 2 H) 4.47-5.09 (m, 3 H) 7.21-9.12 (m, 7 H) | 420 ([M + H]+) |
| 43 | 2 | 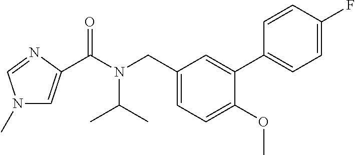 | HCl | 1H NMR (600 MHz, DMSO-d6) d ppm 1.05-1.27 (m, 6 H) 3.68-3.89 (m, 6 H) 4.32-5.05 (m, 3 H) 6.77- 9.00 (m, 9 H) | 382 ([M + H]+) |
| 44 | 1 | 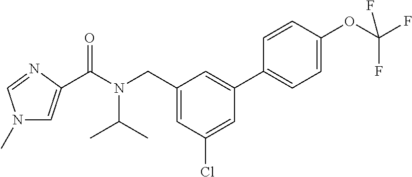 | | 1H NMR (600 MHz, CHLOROFORM-d) d ppm 1.08-1.33 (m, 6 H) 3.63-3.78 (m, 3 H) 4.55-5.86 (m, 3 H) 7.13-7.63 (m, 9 H) | 452 ([M + H]+) |
| 45 | 1 | 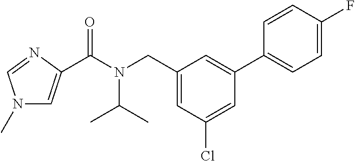 | | 1H NMR (600 MHz, CHLOROFORM-d) d ppm 1.05-1.34 (m, 6 H) 3.56-3.81 (m, 3 H) 4.51-5.86 (m, 3 H) 7.05-7.14 (m, 2 H) 7.28-7.58 (m, 7 H) | 386 ([M + H]+) |
| 46 | 2 | 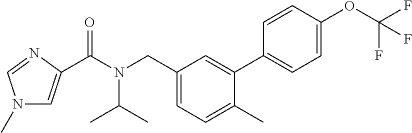 | HCl | 1H NMR (600 MHz, CHLOROFORM-d) d ppm 1.18-1.40 (m, 6 H) 2.23 (s, 3 H) 4.12 (m, 7 H) 6.95-7.74 (m, 8 H) 9.37- 9.67 (m, 1 H) | 432 ([M + H]+) |
| 47 | 2 | 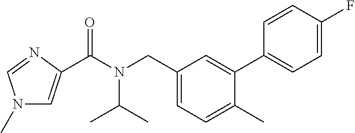 | HCl | 1H NMR (600 MHz, CHLOROFORM-d) d ppm 1.06-1.48 (m, 6 H) 2.22 (br. s, 3 H) 3.76-4.91 (m, 6 H) 6.70-7.78 (m, 8 H) 9.43-9.80 (m, 1 H) | 366 ([M + H]+) |
| 48 | 1 | 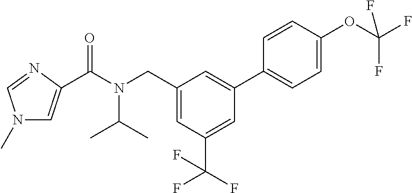 | | 1H NMR (600 MHz, CHLOROFORM-d) d ppm 1.09-1.37 (m, 6 H) 3.71 (m, 3 H) 4.58-5.89 (m, 3 H) 7.10-7.72 (m, 9 H) | 486 ([M + H]+) |

TABLE 1-6

| | | | | | (ESI pos.) |
|---|---|---|---|---|---|
| | Ex- | | | | m/z |
| Com- | am- | | | | (ESI neg.) |
| pound | ple | Structure | Salt | NMR | m/z |
| 49 | 1 | 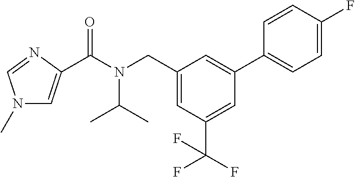 | | 1H NMR (600 MHz, CHLOROFORM-d) d ppm 1.08-1.33 (m, 6 H) 3.60-3.78 (m, 3 H) 4.59-5.86 (m, 3 H) 7.07-7.17 (m, 2 H) 7.33-7.73 (m, 7 H) | 420 ([M + H]+) |
| 50 | 1 | 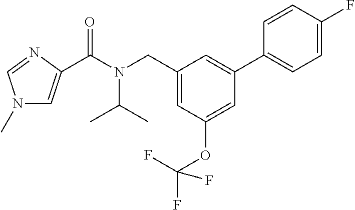 | | 1H NMR (600 MHz, CHLOROFORM-d) d ppm 1.09-1.35 (m, 6 H) 3.59-3.79 (m, 3 H) 4.58-5.87 (m, 3 H) 7.02-7.73 (m, 9 H) | 436 ([M + H]+) |
| 51 | 1 | 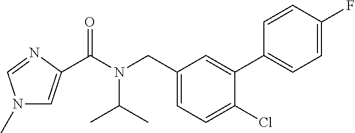 | | 1H NMR (600 MHz, CHLOROFORM-d) d ppm 1.12-1.32 (m, 6 H) 3.62-3.78 (m, 3 H) 4.55-5.82 (m, 3 H) 7.03-7.72 (m, 9 H) | 386 ([M + H]+) |
| 52 | 5 | 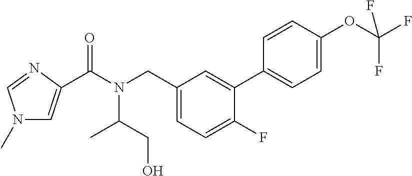 | | 1H NMR (600 MHz, CHLOROFORM-d) d ppm 0.99-1.18 (m, 3 H) 3.50-3.81 (m, 6 H) 4.47-6.41 (m, 4 H) 7.03-7.71 (m, 9 H) | 452 ([M + H]+) |
| 53 | 7 | 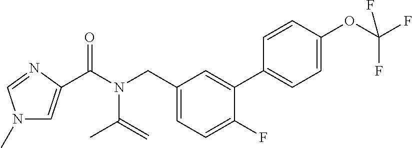 | | 1H NMR (600 MHz, CHLOROFORM-d) d ppm 1.91-2.20 (m, 3 H) 3.68-3.75 (m, 3 H) 4.59-5.00 (m, 4 H) 7.05-7.59 (m, 9 H) | |
| 54 | 1 | 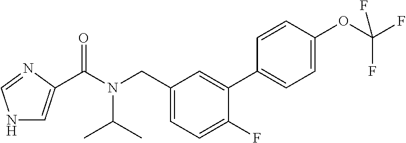 | | 1H NMR (600 MHz, CHLOROFORM-d) d ppm 1.25 (m, 6 H) 4.52-5.85 (m, 3 H) 6.91-7.84 (m, 9 H) | 422 ([M + H]+) 420 ([M − H]−) |

### Test Example 1

### Glycine Uptake Inhibition Experiment

A glycine uptake experiment was conducted as described in Neuron, 8, 927-935, 1992. Glioma T98G cells endogenously expressing human type 1 glycine transporter (GlyT1) were used. T98G cells were seeded in a 96-well plate at 2.0×10⁴ cells/well and cultured overnight in a CO₂ incubator. A test compound was dissolved in a 100% DMSO solution and then dissolved in 10 mM HEPES buffer (pH 7.4) containing 150 mM sodium chloride, 1 mM calcium chloride, 5 mM potassium chloride, 1 mM magnesium chloride, 10 mM glucose and 0.2% bovine serum albumin. After removing the cell culture medium, the cells were pre-treated with the test compound for 10 minutes. Subsequently, the test compound and [³H]glycine (final concentration: 250 nM) were added to the cells and incubated at room temperature for 15 minutes. After the incubation, the extracellular solution was aspirated with a manifold to remove the excess labeled glycine existing outside the cells, and the cells were then lysed with 0.5 M aqueous sodium hydroxide. The amount of glycine uptake was determined by measuring radioactivity in the cell lysate using a liquid scintillation counter. Glycine uptake in the presence of 10 μM ALX5407 was defined as non-specific uptake, and the value calculated by subtracting this non-specific uptake from the total uptake in the absence of 10 μM ALX5407 was defined as specific uptake. Moreover, an inhibition curve was obtained for each test compound at concentrations of 10 to 10 M to calculate the glycine uptake inhibitory activity (IC₅₀ value) of each test substance.

It should be noted that ALX5407 is a HCl salt of N-[(3R)-3-([1,1′-biphenyl]-4-yloxy)-3-(4-fluorophenyl)propyl]-N-methylglycine.

The compounds of the present invention were found to have IC₅₀ values of greater than 1 μM for Compounds 41, 43, 46-50 and 54, and IC₅₀ values of less than 1 μM for the other compounds. More specifically, Compounds 3, 4, 5, 30, 32, 33, 37, 51 and 53 had IC₅₀ values of 0.1 μM or more, and the other compounds had IC₅₀ values of less than 0.1 μM. For example, Compounds 2, 6, 7, 8, 10, 11, 13, 14, 15, 16, 17, 18 19, 20, 21, 22, 23, 24, 25, 26, 28, 31, 34, 35, 36, 39, 40, 42, and 52 were found to have IC₅₀ values of 12.6 nM, 14.6 nM, 25.9 nM, 11.0 nM, 14.8 nM, 35.1 nM, 4.33 nM, 2.78 nM, 21.4 nM, 41.5 nM, 18.8 nM, 8.89 nM 18.6 nM, 7.19 nM, 13.6 nM, 11.6 nM, 21.4 nM, 14.2 nM, 25.4 nM, 40.0 nM, 22.6 nM, 21.0 nM, 49.4 nM, 48.9 nM, 38.2 nM, 48.5 nM, 35.9 nM, 48.3 nM, and 30.7 nM, respectively.

### Test Example 2

### Membrane Permeability Experiment

In the development of drugs, membrane permeability is one of the important factors in relation to the in vivo absorption rate of orally administered drugs, and high membrane permeability contributes to the good absorption from the intestinal tract of drugs (see

Pharmaceutical Research (2002) Vol. 19, No. 7, 921-925).

A membrane permeability test was performed with PAMPA Evolution™ (pION Inc.) according to the protocol recommended by pION Inc. Specifically, a test compound solution (i.e., a DMSO solution of a test compound, which was further diluted with system solution adjusted to each pH value (4.0, 5.0, 6.2, or 7.4)) was prepared and added to the Donor side of the sandwich plate separated by a artificial lipid bilayer (GIT-0). To the Acceptor side, acceptor sink buffer was added. After a given period of time, the Donor and Acceptor solutions were analyzed by UV analysis to determine the accumulated amount of the compound, which was then used to calculate the membrane permeability coefficient Pe (×10⁻⁶ cm/sec), whereby the compound was evaluated for its membrane permeability. As a result, Compound Nos. 8, 10, 12, 15, 16, 18-21, 23-26, 28, 34-36, 39-42 and 54 according to the present invention each showed good membrane permeability that was higher than the membrane permeability coefficient of metoprolol, a highly permeable marker compound.

### Test Example 3

### Test for Substrate Recognition by P-gp

In the case of drugs that act on the central nervous system, it is generally important for such drugs to be transferred from blood into brain for development of their efficacy. In the blood-brain barrier, there is found P-glycoprotein (P-gp), which is a typical member of the efflux transporters that control drug penetration; and P-gp inhibits the penetration of its substrate drugs into the brain. Thus, in developing a drug, recognition performance as a P-gp substrate can be used as an indicator of brain penetration.

A test for substrate recognition by P-gp was conducted as described in J. Pharmacol. Exp. Ther. (1992) Vol. 263, No. 2, 840-845 and J. Biol. Chem. (1992) Vol. 267, No. 34, 24248-24252. Specifically, LLC-GA5-COL300 cells (i.e., a Human MDR1-expressing system originating from a pig kidney-derived cultured renal epithelial cell line, LLC-PK1) were used after being cultured for 4 days on trans wells, and the medium in each well was replaced by Hank's balanced salt solution (HBSS) immediately before use in the test. After a test compound solution (i.e., a DMSO solution of a test compound, which was further diluted with HBSS and adjusted to a final concentration of 10 μM) was added to the Donor side of the LLC-GA5-COL300 cells, aliquots of HBSS were sampled over time from the Acceptor side to determine the test compound concentration in each collected sample by LC-MS/MS.

The membrane permeability coefficient (×10⁻⁶ cm/sec) was calculated by the accumulated amount of the compound into the Acceptor side for both directions, Apical to Basolateral and Basolateral to Apical. and the ratio of these coefficients (Efflux Ratio) was then used to evaluate substrate recognition by P-gp.

As a result, Compound Nos. 10, 17, 18, 20, 22, 24, 36, and 54 were each determined not to be recognized as a P-gp substrate, as evaluated by the criteria described in Nature Reviews Drug Discovery (2010), Vol. 9, 215-236, thus suggesting that these compounds would have good brain penetration (see Pharmaceutical Research (2001), Vol. 18, No. 12, 1660-1668). From this result, the compounds of the present invention are expected to be effective for use as drugs that act on the central nervous system.

### Test Example 4

### Social Recognition Test

This experiment was performed using male Sprague-Dawley rats according to the reported method (Shimazaki et al., Psychopharmacology, 209, 263-270, 2010). Adult rats (9 weeks old) received intraperitoneal administration of MK-801 (0.1 mg/kg), and were immediately placed in test cages and acclimated for 30 minutes. Thereafter, juvenile rats (4 weeks old) were placed in the same test cages, where juvenile rats and adult rats were left for 5 minutes, during which the time it took for the adult rats to show social behavior (sniffing, grooming, following) to the juvenile rats was measured (first exploration time). Then, the juvenile rats were removed from the test cages and returned to their home cages. After 30 minutes, the same juvenile rats that were used in the first exploration were placed in the test cages, and the time it took for the adult rats to show social behavior (sniffing, grooming, following) to the juvenile rats during a 5-minute period was measured (second exploration time). The social recognition was expressed as the ratio of second exploration time to first exploration time. The test substance (Compound 10) was orally administered one hour before the start of first exploration. The results are shown below. The vehicle group was administered a 0.5% methyl cellulose solution.

| | Ratio |
|---|---|
| | (second exploration time/ |
| | first exploration time) |
| Vehicle group | 0.87 ± 0.06 |
| Test substance (0.03 mg/kg) group | 0.72 ± 0.04 |
| Test substance (0.1 mg/kg) group | 0.65 ± 0.05 ^{p<0.05} |
| Test substance (0.3 mg/kg) group | 0.60 ± 0.06 ^{p<0.01} |

n = 15-16, statistical significance was analyzed by ANOVA followed by Dunnett's test (parametric)

Compared to the vehicle group, the test substance groups showed significant reductions in the ratio of second exploration time to first exploration time, indicating that the test substance had an enhancing effect on cognitive functions.

### INDUSTRIAL APPLICABILITY

The compounds of the present invention have an inhibitory effect against type 1 glycine transporter (GlyT1) and are thus effective for prevention or treatment of glycine transporter-related diseases, more specifically schizophrenia, Alzheimer's disease, cognitive impairment, dementia, anxiety disorders (e.g., generalized anxiety disorder, panic disorder, obsessive-compulsive disorder, social anxiety disorder, post-traumatic stress disorder, specific phobias, acute stress disorder), depression, drug dependence, convulsion, tremor, pain, and sleep disorders, etc.

1. A compound represented by formula [I] or a pharmaceutically acceptable salt thereof: 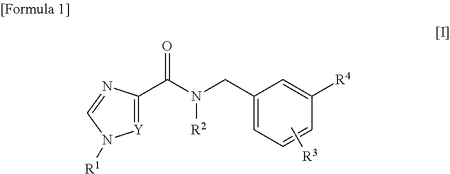
wherein
R¹ represents a hydrogen atom or a C₁₋₆ alkyl group,
R² represents a C₁₋₆ alkyl group, a C₂₋₆ alkenyl group, a C₃₋₆ cycloalkyl group, a C₁₋₆ haloalkyl group, a C₁₋₆ hydroxyalkyl group, or a C₁₋₆ alkoxy-C₁₋₆ alkyl group,
R³ represents a hydrogen atom, a C₁₋₆ alkyl group, a C₁₋₆ alkoxy group, a C₁₋₆ haloalkyl group, a C₁₋₆ haloalkoxy group, or a halogen atom,
R⁴ represents a phenyl group, which may be substituted with 1 to 3 substituents selected from the group consisting of a C₁₋₆ alkyl group, a C₁₋₆ alkoxy group, a C₁₋₆ haloalkyl group, a C₁₋₆ haloalkoxy group, a C₁₋₆ hydroxyalkyl group, a C₁₋₆ alkoxy-C₁₋₆ alkyl group, a C₂₋₇ alkoxycarbonyl group, a cyano group, and a halogen atom, and
Y represents the formula CH or a nitrogen atom.

2. The compound or pharmaceutically acceptable salt thereof according to claim 1, wherein R² is a branched C₃₋₆ alkyl group or a C₃₋₆ cycloalkyl group.

3. The compound or pharmaceutically acceptable salt thereof according to claim 1, wherein Y is the formula CH.

4. The compound or pharmaceutically acceptable salt thereof according to claim 1, wherein R³ is a halogen atom.

5. The compound or pharmaceutically acceptable salt thereof according to claim 1, wherein R³ is a fluorine atom.

6. The compound or pharmaceutically acceptable salt thereof according to claim 1, wherein R⁴ is a phenyl group, or a phenyl group substituted with 1 to 3 substituents selected from the group consisting of a C₁₋₆ alkoxy group, a C₁₋₆ haloalkoxy group, a C₁₋₆ hydroxyalkyl group, and a halogen atom.

7. The compound or pharmaceutically acceptable salt thereof according to claim 1, wherein the compound of formula [I] is represented by the following formula: 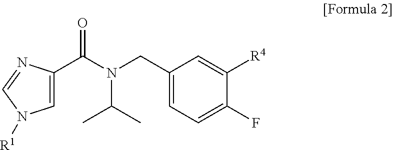
wherein R¹ represents a methyl group or an ethyl group, and R⁴ is a phenyl group, or a phenyl group substituted with 1 to 3 substituents selected from the group consisting of a C₁₋₆ alkoxy group, a C₁₋₆ haloalkoxy group, a C₁₋₆ hydroxyalkyl group, and a halogen atom.

8. The compound or pharmaceutically acceptable salt thereof according to claim 1, wherein the compound is
N-{[6-fluoro-4′-(trifluoromethoxy)biphenyl-3-yl]methyl}-1-methyl-N-(propan-2-yl)-1H-imidazole-4-carboxamide,
N-{[6-fluoro-3′-(trifluoromethoxy)biphenyl-3-yl]methyl}-1-methyl-N-(propan-2-yl)-1H-imidazole-4-carboxamide,
1-methyl-N-(propan-2-yl)-N-[(3′,4′,6-trifluorobiphenyl-3-yl)methyl]-1H-imidazole-4-carboxamide,
N-[(4′,6-difluorobiphenyl-3-yl)methyl]-1-methyl-N-(propan-2-yl)-1H-imidazole-4-carboxamide,
N-[(3′,6-difluoro-4′-methoxybiphenyl-3-yl)methyl]-1-methyl-N-(propan-2-yl)-1H-imidazole-4-carboxamide,
N-[(3′,6-difluorobiphenyl-3-yl)methyl]-1-methyl-N-(propan-2-yl)-1H-imidazole-4-carboxamide,
N-[(6-fluorobiphenyl-3-yl)methyl]-1-methyl-N-(propan-2-yl)-1H-imidazole-4-carboxamide,
N-[(6-fluoro-3′-methoxybiphenyl-3-yl)methyl]-1-methyl-N-(propan-2-yl)-1H-imidazole-4-carboxamide,
N-[(6-fluoro-4′-methoxybiphenyl-3-yl)methyl]-1-methyl-N-(propan-2-yl)-1H-imidazole-4-carboxamide,
N-[(4′-chloro-6-fluorobiphenyl-3-yl)methyl]-1-methyl-N-(propan-2-yl)-1H-imidazole-4-carboxamide,
1-methyl-N-(propan-2-yl)-N-[(3′,5′,6-trifluorobiphenyl-3-yl)methyl]-1H-imidazole-4-carboxamide,
N-{[3′-(difluoromethoxy)-6-fluorobiphenyl-3-yl]methyl}-1-methyl-N-(propan-2-yl)-1H-imidazole-4-carboxamide,
N-{[4′-(difluoromethoxy)-6-fluorobiphenyl-3-yl]methyl}-1-methyl-N-(propan-2-yl)-1H-imidazole-4-carboxamide,
1-methyl-N-(propan-2-yl)-N-[(3′,4′,5′,6-tetrafluorobiphenyl-3-yl)methyl]-1H-imidazole-4-carboxamide, or
1-ethyl-N-{[6-fluoro-4′-(trifluoromethoxy)biphenyl-3-yl]methyl}-N-(propan-2-yl)-1H-imidazole-4-carboxamide.

9. The compound or pharmaceutically acceptable salt thereof according to claim 1, wherein the compound is
N-{[6-fluoro-4′-(trifluoromethoxy)biphenyl-3-yl]methyl}-1-methyl-N-(propan-2-yl)-1H-imidazole-4-carboxamide,
N-{[6-fluoro-3′-(trifluoromethoxy)biphenyl-3-yl]methyl}1-methyl-N-(propan-2-yl)-1H-imidazole-4-carboxamide,
1-methyl-N-(propan-2-yl)-N-[(3′,4′,6-trifluorobiphenyl-3-yl)methyl]-1H-imidazole-4-carboxamide,
N-[(4′,6-difluorobiphenyl-3-yl)methyl]-1-methyl-N-(propan-2-yl)-1H-imidazole-4-carboxamide,
N-{[3′-(difluoromethoxy)-6-fluorobiphenyl-3-yl]methyl}1-methyl-N-(propan-2-yl)-1H-imidazole-4-carboxamide,
N-{[4′-(difluoromethoxy)-6-fluorobiphenyl-3-yl]methyl}1-methyl-N-(propan-2-yl)-1H-imidazole-4-carboxamide,
1-methyl-N-(propan-2-yl)-N-[(3′,4′,5′,6-tetrafluorobiphenyl-3-yl)methyl]-1H-imidazole-4-carboxamide, or
1-ethyl-N-{[6-fluoro-4′-(trifluoromethoxy)biphenyl-3-yl]methyl}N-(propan-2-yl)-1H-imidazole-4-carboxamide.

10. A pharmaceutical preparation, which comprises the compound or pharmaceutically acceptable salt thereof according to claim 1 as an active ingredient.

11. A prophylactic or therapeutic agent for diseases of schizophrenia, Alzheimer's disease, cognitive impairment, dementia, anxiety disorders, depression, drug dependence, convulsion, tremor, or sleep disorders, which comprises the compound or pharmaceutically acceptable salt thereof according to claim 1 as an active ingredient.
